(19) United States
(12) Patent Application Publication
(10) Pub. No.: US 2007/0025957 A1
(43) Pub. Date: Feb. 1, 2007

(54) Vascular targeting of ocular neovascularization

(76) Inventors: Michael G. Rosenblum, Sugar Land, TX (US); Peter A. Campochiaro, Baltimore, MD (US)

Correspondence Address: FULBRIGHT & JAWORSKI L.L.P., 600 CONGRESS AVE., SUITE 2400, AUSTIN, TX, 78701, US

(21) Appl. No.: 11/414,782

(22) Filed: Apr. 28, 2006

Related U.S. Application Data
(60) Provisional application No. 60/675,958, filed on Apr. 29, 2005.

(51) Int. Cl. A61K 39/395; A61K 39/02; A61K 38/21; A61K 36/47; A61K 38/16; A61K 38/20; A61K 38/19

(52) U.S. Cl. 424/85.1; 514/12; 424/145.1; 424/236.1; 424/731; 424/85.2; 424/85.4; 514/8

(57) ABSTRACT

Methods of treating or preventing eye disease in a subject, involving administering to the subject a therapeutically effective amount of a pharmaceutical composition comprising a polypeptide having a vascular endothelial targeting amino acid sequence and a cytotoxic amino acid sequence are disclosed. For example, the vascular endothelial targeting amino acid sequence may be a VEGF sequence, such as a VEGF₁₂₁ sequence. Exemplary cytotoxic amino acid sequences include toxin sequences, such as gelonin, pro-apoptotic sequences, and anti-angiogenic sequences. The eye disease may be any eye disease, such as an eye disease associated with choroidal neovascularization, retinal neovascularization, iris neovascularization, or corneal neovascularization.

The present application is related to U.S. Provisional Patent Application 60/675,958, filed on Apr. 28, 2005, hereby incorporated by reference in its entirety.

The government owns rights in the present invention pursuant to grant number EY05951 from the National Institutes of Health.

### BACKGROUND OF THE INVENTION

1. Field of the Invention

The present invention relates generally to the fields of ophthalmology, protein chemistry, and toxicology. More particularly, the present invention relates to methods of treating or preventing eye disease in a subject that involve administering to the subject a therapeutically effective amount of a pharmaceutical composition comprising a polypeptide having a vascular endothelial targeting amino acid sequence and a cytotoxic amino acid sequence. Exemplary eye diseases to be treated or prevented include choroidal neovascularization (CNV) due to any cause including but not limited to age-related macular degeneration, ocular histoplasmosis, pathologic myopia, and angioid streaks. It also applies to retinal neovascularization of any cause including but not limited to proliferative diabetic retinopathy, retinal vein occlusions, and retinopathy of prematurity. It also applies to iris neovascularization and corneal neovascularization of any causes.

2. Description of Related Art

Diseases complicated by vascular leakage and/or neovascularization in the eye are responsible for the vast majority of visual morbidity and blindness in developed countries. Retinal neovascularization occurs in ischemic retinopathies such as diabetic retinopathy and is a major cause of visual loss in working age patients (Klein et al., 1984). Choroidal neovascularization occurs as a complication of age-related macular degeneration and is a major cause of visual loss in elderly patients (Ferris et al., 1984). Improved treatments are needed to reduce the high rate of visual loss, and their development is likely to be facilitated by greater understanding of the molecular pathogenesis of ocular neovascularization.

The molecular signals that control vascular permeability and neovascularization in the eye are actively being investigated. Members of the vascular endothelial growth factor (VEGF) family control pathological angiogenesis and increased vascular permeability in important eye diseases such as diabetic retinopathy (DR) and age-related macular degeneration (AMD) (reviewed in Witmer et al., 2003).

VEGF has been shown to have numerous functions in disorders associated with neovascularization. It enhances endothelial cell proliferation, migration, and survival and is essential for blood vessel formation. Other roles of VEGF include wound healing, vascular permeability and the regulation of blood flow.

Through alternative splicing of RNA, human VEGF exists as at least four isoforms of 121, 165, 189, or 206 amino acids. The lowest molecular weight isoform, designated VEGF₁₂₁, is a non-heparan sulfate-binding isoform that exists in solution as a disulfide-linked homodimer. VEGF₁₂₁, has been shown to contain the full biological activity of the larger variants.

In humans, the angiogenic actions of VEGF are mediated through two related receptor tyrosine kinases, kinase domain receptor (KDR) and FLT-1. Both are largely restricted to vascular endothelial cells. The receptors for VEGF thus seem to be excellent targets for the development of therapeutic agents that inhibit neovascularization.

Several lines of evidence have suggested that vascular endothelial growth factor (VEGF) is an important stimulator for both retinal and choroidal neovascularization (Aiello et al., 1994; Adamis et al., 1994; Aiello et al., 1995; Adamis et al., 1996; Seo et al., 1999; Ozaki et al., 2000; Kwak et al., 2000; Saishin et al., 2003). This has led to clinical trials testing the effect of VEGF antagonists in patients with subfoveal choroidal neovascularization. Intraocular injections of pegaptanib, an aptamer that binds VEGF, every 6 weeks for 1 year reduced loss of vision compared to sham injections (Gragoudas et al., 2004). Slowing visual loss is an important achievement, but it is not the ultimate goal, which is to improve vision and/or maintain it within a range that permits optimal functioning. Studies are underway to evaluate Ranibizumab, a human monoclonal antibody fragment designed to bind all forms of VEGF, in the treatment of neovascular AMD (Gaudreault et al., 2005).

In animal models, VEGF antagonists are very good at suppressing growth of neovascularization and reducing excessive leakage (Aiello et al., 1995; Adamis et al., 1996; Seo et al., 1999; Ozaki et al., 2000; Kwak et al., 2000; Saishin et al., 2003), but fail to cause regression of new vessels. This is supported by observations in patients with choroidal neovascularization treated with VEGF antagonists in whom leakage is reduced, but the choroidal neovascularization is not eliminated. Regression of neovascularization is likely to be needed to achieve optimal results.

Differentially expressed gene products provide a means to target therapeutic agents to vasculature, a strategy that is commonly referred to as “vascular targeting” (Denekamp, 1984; Denekamp, 1999; Thorpe, 2004). VEGF receptors have been shown to be present in low levels in normal endothelial cells, but substantially more abundant in endothelium of tumor vessels. Vessel markers that have been exploited and demonstrated to have therapeutic potential in tumor models include (but are not limited to), αᵥβ₃ and αᵥβ₅ integrins (Pasqualini et al., 1997), VEGF receptors (Ramakrishnan et al., 1996; Arora et al., 1999; Veenendaal et al., 2002; Liu et al., 2003), the ED-B domain of fibronectin (Nilsson et al., 2001), VCAM-1 (Ran et al., 1998), and PSA (Liu et al., 2002). Endothelial cells participating in angiogenesis in disease processes other than, tumors also display differential gene expression. For instance, there is substantial upregulation of αᵥβ₃ in ischemia-induced retinal neovascularization (Luna et al., 1996).

Molecular engineering has enabled the synthesis of novel chimeric molecules having therapeutic potential. Striking regression of tumors has been achieved in several mouse models by systemic injection of chimeric proteins consisting of a toxin coupled to a homing protein that binds to a gene product that is differentially expressed in tumor vasculature (Pasqualini et al., 1997; Ramakrishnan et al., 1996; Arora et al., 1999; Veenendaal et al., 2002; Liu et al., 2003; Nilsson et al., 2001; Ran et al., 1998; Liu et al., 2002). It has also been showed that a chemical conjugate of VEGF and truncated diphtheria toxin has impressive cytotoxic activity on cell lines expressing receptors for vascular endothelial growth factor. Chimeric fusion constructs targeting the IL-2 receptor, the EGF receptor, and other growth factor/cytokine receptors have been described.

There is the need for improved therapies of neovascular disease affecting the eye. Targeted therapies wherein a vascular targeting agent is employed to target a therapeutic agent to ocular tissue have not been described. Such therapies would not only be beneficial in guiding therapy directly to diseased tissue, but would serve to minimize systemic toxicity and toxicity to healthy ocular tissue, a factor of key importance in the treatment of eye disease.

### SUMMARY OF THE INVENTION

The present inventors have identified a novel form of therapy of ocular disease that involves targeting of certain therapeutic agents to vascular tissue. In particular, they have discovered that certain chimeric fusion constructs, which include a vascular endothelial targeting amino acid sequence and a cytotoxic amino acid sequence, are effective treatments of ocular neovascularization. For example, they have found that intravenous or intraocular administration of a VEGF/gelonin fusion construct causes regression of neovascularization in three animal models of ocular neovascularization (laser-induced rupture of Bruch's membrane, rho/VEGF transgenic mice, and oxygen-induced ischemic retinopathy). The vascular targeting strategy employing these fusion constructs can be applied in the treatment of any eye disease associated with neovascularization, including both malignant and non-malignant neovascular diseases of the eye. Exemplary non-malignant eye diseases include choroidal neovascularization (CNV) due to any cause including but not limited to age-related macular degeneration, ocular histoplasmosis, pathologic myopia, and angioid streads. The novel forms of therapy can also be applied in the treatment of retinal neovascularization of any cause including but not limited to proliferative diabetic retinopathy, retinal vein occlusions, and retinopathy of prematurity. It also applies to iris neovascularization and corneal neovascularization of any causes.

The present invention generally pertains to methods of treating or preventing eye disease in a subject, comprising administering to the subject a therapeutically effective amount of a pharmaceutical composition comprising a polypeptide having a vascular endothelial targeting amino acid sequence and a cytotoxic amino acid sequence which results in treatment or prevention of eye disease in the subject. The polypeptide may or linker between the vascular endothelial targeting amino acid sequence and the cytotoxic amino acid sequence. The linker would be any linker known to those of ordinary skill in the art. Exemplary linkers include G₄S, (G₄S)₂, (G₄S)₃, 218 linker, an enzymatically cleavable linker, or a pH cleavable linker.

A vascular endothelial targeting sequence, as discussed in greater detail elsewhere in this specification, is defined herein to refer to any amino acid sequence that has the capability to bind (covalently or non-covalently) or otherwise attach to an endothelial cell of a blood vessel. Any vascular endothelial targeting amino acid sequence is contemplated for inclusion in the methods of the present invention. For example, the vascular endothelial targeting sequence may be VEGF, FGF, integrin, fibronectin, I-CAM, PDGF, or an antibody to a molecule expressed on the surface of a vascular endothelial cell. Any VEGF amino acid sequence is contemplated by the present invention. For example, the VEGF sequence may be an isoform selected from the group consisting of VEGF₁₂₁, VEGF₁₆₅, VEGF₁₈₉, and VEGF₂₀₆. In certain particular embodiments, the VEGF isoform is VEGF₁₂₁, or a VEGF sequence selected from the group consisting of SEQ ID NOs:4-10.

A cytotoxic amino acid sequence is defined herein to refer to any amino acid sequence that is capable of causing injury or death to a cell. Any cytotoxic amino acid sequence known to those of ordinary skill in the art is contemplated for inclusion in the methods of the present invention. In certain embodiments of the present invention, the cytotoxic amino acid sequence is a toxin. For example, the toxin may be a ribosome inactivating protein (RIP). Exemplary ribosome inactivating proteins include gelonin, maize RIP, saporin, ricin, ricin A chain, barley RIP, momordin, alpha-momorcharin, beta-momorcharin, Shiga-like RIP, and a-sarcin. Additional exemplary toxins include abrin, an aquatic-derived cytotoxin, Pseudomonas exotoxin, a DNA synthesis inhibitor, a RNA synthesis inhibitor, a prodrug, a light-activated porphyrin, trichosanthin, tritin, pokeweed antiviral protein, mirabilis antiviral protein (MAP), Dianthin 32, Dianthin 30, bryodin, shiga, diphtheria toxin, diphtheria toxin A chain, dodecandrin, tricokirin, bryodin, and luffin.

In further embodiments of the present invention, the cytotoxic amino acid sequence is an anti-angiogenic amino acid sequence. Any anti-angiogenic amino acid sequence known to those of ordinary skill in the art is contemplated for inclusion in the methods of the present invention. For example, the anti-angiogenic amino acid sequence may be TIMP-1, TIMP-2, TIMP-3, TIMP-4, endostatin, angiostatin, endostatin XVIII, endostatin XV, the C-terminal hemopexin domain of matrix metalloproteinase-2, the kringle 5 domain of human plasminogen, the monokine-induced by interferon-gamma (Mig), the interferon-alpha inducible protein 10 (IP10), soluble FLT-1 (fms-like tyrosine kinase 1 receptor), or kinase insert domain 15 receptor (KDR).

In some embodiments of the present invention, the cytotoxic amino acid sequence induces apoptosis. Exemplary cytotoxic sequences that are capable of inducing apoptosis include granzyme B, Bax, TNF-α, TNF-β, TGF-β, IL-12, IL-3, IL-24, IL-18, TRAIL, IFN-α, INF-β, IFN-γ, a Bcl protein, Fas ligand, and a caspase. One of ordinary skill in the art would be familiar with these and other cytotoxic amino acid sequences capable of inducing apoptosis.

The subject can be any subject, such as a mammal or avian species. In certain particular embodiments of the present invention, the subject is a human. The subject may or may not be currently affected by an eye disease. In some embodiments, the subject is a subject at risk of developing an eye disease.

Any eye disease is contemplated for treatment and prevention by the methods of the present invention. In particular embodiments of the present invention, the eye disease is an eye disease associated with neovascularization. Neovascularization is defined herein to refer to proliferation of blood vessels in tissue not normally containing them, or proliferation of blood vessels of a different kind than usual in tissue. One of ordinary skill in the art would be familiar with the wide range of ophthalmic conditions associated with neovascularization.

The neovascularization may include retinal neovascularization, choroidal neovascularization, or other ophthalmic neovascularization. Exemplary eye diseases associated with neovascularization include age-related macular degeneration, diabetic retinopathy, proliferative vitreoretinopathy, and ophthalmic tumors. The ophthalmic tumor, for example, may be a choroidal melanoma, a retinoblastoma, a metastatic tumor, or a uveal melanoma.

Any method of administration of the pharmaceutical compositions known to those of ordinary skill in the art is contemplated by the present invention. In certain embodiments, the composition is administered intravascularly. In further embodiments, the composition is administered intraocularly. Any method of intraocular administration known to those of ordinary skill in the art is contemplated for inclusion in the present invention. For example, the intraocular administration may be by intravitreal administration, administration into the anterior chamber, or administration into an intraocular tumor.

In certain particular embodiments of the present invention, the subject has neovascularization secondary to age-related macular degeneration and is administered a fusion protein of VEGF₁₂₁, and recombinant gelonin by intravitreal administration. Any therapeutic amount of the fusion protein is contemplated by the present methods. For example, in some embodiments, between about 0.5 ng and about 10 ng of the fusion protein is administered intravitreally. In further embodiments, between about 1 ng and about 4 ng of the fusion protein is administered intravitreally.

Administration of the pharmaceutical composition may involve a single administration, or more than one administration. The method of treating or preventing eye disease may be administrated as the sole therapeutic or preventive agent to the subject, or it may be administered in combination with other methods to treat or prevent eye disease. One of ordinary skill in the art would be familiar with other methods of treatment or prevention of eye disease that can be applied in combination with the present therapeutic methods. Exemplary forms of therapy include oral therapy, topical therapy, intraocular therapy, laser photocoagulation, cryotherapy, radiation therapy, surgical therapy, gene therapy, and immunotherapy.

In other embodiments of the present invention, the methods set forth above further involve identifying a patient in need of such therapy. For example, the patient may be a patient with an eye disease, such as an eye disease associated with neovascularization. Alternately, the patient may be a patient at risk of developing an eye disease, such as an eye disease associated with neovascularization.

Further embodiments of the present invention pertain to methods of treating or preventing an eye disease associated with neovascularization in a subject that involve intraocularly administering to the subject a therapeutically effective amount of a composition comprising a polypeptide having a vascular endothelial targeting amino acid sequence and a cytotoxic amino acid sequence, wherein the eye disease is prevented or treated. As set forth above, the polypeptide may or may not include a linker between the vascular endothelial targeting amino acid sequence and the cytotoxic amino acid sequence. Exemplary linkers include those linkers set forth above.

The vascular endothelial targeting amino acid sequence may include any of the sequences set forth above. In certain particular embodiments, the vascular endothelial targeting amino acid sequence is a VEGF sequence, such as VEGF₁₂₁ or a VEGF sequence selected from the group consisting of SEQ ID NOs:4-10.

The cytotoxic amino acid sequence may include any of those cytotoxic amino acid sequences set forth above. For example, the cytoxic amino acid sequence may be a toxin, such as a ribosome inactivating protein or an amino acid sequence that induces apoptosis.

As set forth above, the subject can be any subject, such as a mammal or avian species. In certain particular embodiments, the subject is a human. As discussed above, the eye disease can be any eye disease, such as an eye disease associated with neovascularization. Eye diseases associated with neovascularization have been discussed above, and are discussed in greater detail elsewhere in this specification.

As set forth above, any method of administration known to those of ordinary skill in the art is contemplated by the methods of the present invention. For example, administration may be intraocular. Intraocular administration can be by any method known to those of ordinary skill in the art, such as intravitreal administration, administration into the anterior chamber, or administration into an intraocular tumor.

In some embodiments of the present invention, the subject has neovascularization secondary to age-related macular degeneration and is administered a fusion protein of VEGF₁₂₁, and recombinant gelonin by intravitreal administration. Any amount of the pharmaceutical composition of the fusion protein may be administered. For example, in some embodiments, between about 0.5 ng and about 10 ng of the fusion protein is administered intravitreally. In further embodiments, between about 1 ng and about 4 ng of the fusion protein is administered intravitreally.

As set forth above, the pharmaceutical composition may be administered once, or more than once. Further, the methods set forth herein may further comprise treating the subject with other eye therapy, such as any of those forms of therapy set forth above. For example, the other therapy may be oral therapy, topical therapy, intraocular therapy, laser photocoagulation, cryotherapy, radiation therapy, surgical therapy, gene therapy, or immunotherapy, or more than one of these forms of therapy. In further embodiments, the methods set forth herein further involve identifying a patient in need of such therapy.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-F. Localization of VEGF/recombinant gelonin (rGel) in choroidal neovascularization after intravenous injection. Adult C57BL/6 mice had laser-induced rupture of Bruch's membrane in each eye and after one week they were given a tail vein injection of PBS (A and B), 45 mg/kg of rGel (C and D), or 45 mg/kg of VEGF/rGel (E and F). One hour after the intravenous injections, mice were euthanized and eyes were removed and snap frozen. Ocular sections were histochemically stained with Griffonia simplicifolia lectin (GSA) visualized with diaminobenzidine (A, C, and E) or immunohistochemically stained with anti-gelonin antibody using a fluorescein-labeled secondary antibody. (A) An ocular section of a mouse given an intravenous injection of PBS one week after rupture of Bruch's membrane shows GSA-stained choroidal neovascularization (arrows) at a rupture site. (B) A section adjacent to the one shown in (A) immunofluorescently stained with anti-gelonin shows faint background staining throughout the retina with no increase in staining in the choroidal neovascularization (arrows). (C) An ocular section of a mouse given an intravenous injection of rGel one week after rupture of Bruch's membrane shows GSA-stained choroidal neovascularization (arrows) at a rupture site. (D) A section adjacent to the one shown in (C) immunofluorescently stained with anti-gelonin shows faint background staining throughout the retina with no increase in staining in the choroidal neovascularization (arrows). (E) An ocular section of a mouse given an intravenous injection of VEGF/rGel one week after rupture of Bruch's membrane shows GSA-stained choroidal neovascularization (arrows) at a rupture site. (F) A section adjacent to the one shown in (E) immunofluorescently stained with anti-gelonin shows faint background staining in the retina with staining above background throughout the choroidal neovascularization (arrows), indicating localization of the VEGF/rGel within the choroidal neovascularization.

FIG. 2A-E. Intravenous injection of VEGF/rGel causes regression of choroidal neovascularization. Thirty adult C57BL/6 mice had laser-induced rupture of Bruch's membrane at three locations in each eye. After 1 week, 7 mice were perfused with fluorescein-labeled dextran and the baseline amount of choroidal neovascularization at rupture sites was measured by image analysis of choroidal flat mounts (A). The remaining mice were divided into three groups: 8 mice received a tail vein injection of 45 mg/kg of rGel (B), 7 mice received a tail vein injection of PBS (C), and 8 mice received a tail vein injection of 45 mg/kg of VEGF/rGel (D). After 1 week, the mice were perfused with fluorescein-labeled dextran and choroidal flat mounts were examined by fluorescence microscopy. The area of choroidal neovascularization at rupture sites appeared substantially smaller in mice that had been injected with VEGF/rGel (D, arrows) than those in mice that had been injected with rGel (B, arrows) or PBS (C, arrows). It was also smaller than the amount of choroidal neovascularization seen at baseline (A). Image analysis confirmed that the area of choroidal neovascularization was significantly smaller one week after injection of VEGF/rGel compared to baseline (E).

*p=0.0031 for difference from baseline by linear mixed model

^{†}p<0.0001 for difference from gelonin or PBS by linear mixed model

Bar=100 μm

FIG. 3A-E. Intravitreous injection of VEGF/rGel causes regression of choroidal neovascularization. Forty adult C57BL/6 mice had laser-induced rupture of Bruch's membrane at three locations in each eye. After 1 week, 10 mice were perfused with fluorescein-labeled dextran and the baseline amount of choroidal neovascularization at rupture sites was measured by image analysis of choroidal flat mounts. The remaining mice were divided into 2 groups: 9 mice received an intravitreous injection 5 ng of rGel in one eye and PBS in the fellow eye, and 13 mice received an intravitreous injection of 5 ng of VEGF/rGel in one eye and PBS in the fellow eye. After 1 week, the mice were perfused with fluorescein-labeled dextran and choroidal flat mounts were examined by fluorescence microscopy. There was a substantial amount of baseline choroidal neovascularization at 7 days after rupture of Bruch's membrane (A, arrows). At day 14, seven days after injection of rGel (B, arrows) or PBS (C, arrows), the area of choroidal neovascularization at rupture sites appeared similar to that seen at baseline (A). The amount of choroidal neovascularization seen 7 days after injection of VEGF/rGel (D, arrows) appeared less than that seen after injection of rGel or PBS, and less than that seen at baseline. Image analysis confirmed that the area of choroidal neovascularization was significantly smaller one week after injection of VEGF/rGel compared to injection of rGel or PBS, or the baseline amount (E).

*p<0.0001 for difference from baseline by linear mixed model

^{†}p=0.0007 for difference from gelonin or PBS by linear mixed model

Bar=100 μm

FIG. 4A-C. Intravitreous injection of VEGF/rGel causes regression of subretinal neovascularization in rho/VEGF transgenic mice. Several litters of hemizygous rho/VEGF transgenic mice were divided into 2 groups. The first group (n=8) was perfused with fluorescein-labeled dextran at P21 and the baseline amount of neovascularization on the outer surface of the retina was measured by fluorescence microscopy and image analysis of retinal flat mounts. The second group (n=9) received an intravitreous injection of 5 ng of rGel in one eye and 5 ng of VEGF/rGel in the fellow eye at P21. At P25, the mice were perfused with fluorescein labeled dextran and the area of neovascularization on the outer surface of the retina was measured. (A) High magnification view of a retinal flat mount of a rho/VEGF mouse at P21 shows numerous tufts of neovascularization (arrows) partially surrounded by RPE cells. The retinal vessels are out-of-focus in the background. (B) At P25, a retinal flat mount from an eye that received an intravitreous injection of rGel at P21 shows several tufts of neovascularization (arrows). (C) At P25, a retinal flat mount from an eye that received an intravitreous injection of VEGF/rGel at P21, shows only one small remaining bud of neovascularization (arrow).

*p=0.0032 for difference from baseline by linear mixed model

^{†}p=0.0193 for difference from gelonin by linear mixed model

Bar=1100 μm

FIG. 5A-G. Intravitreous injection of VEGF/rGel causes regression of ischemia-induced retinal neovascularization. Mice were placed in 75% oxygen at P7 and at P12 they were removed to room air. At P17, the baseline amount of neovascularization was measured (n=6) and the remaining mice (n=7) were given an intravitreous injection of 5 ng of VEGF/rGel in one eye and 5 ng of rGel in the fellow eye. At P21, ocular sections stained with Griffonia simplicifolia lectin showed prominent baseline neovascularization on the surface of the retina in P17 mice (A and B, arrows). Substantial neovascularization was also seen at P21 in eyes that had been injected with rGel (C and D, arrows), but almost no neovascularization was detectable in eyes that had been injected with VEGF/rGel (E and F, arrow). Measurement of the area of retinal neovascularization by image analysis showed that at P21 eyes injected with VEGF/rGel had less neovascularization than that seen in eyes injected with rGel and less than the baseline neovascularization at P17 (G). *p=0.0004 for difference from baseline by linear mixed model; ^{†}p=0.0017 for difference from gelonin by linear mixed model; Bar=100 μm.

### DESCRIPTION OF ILLUSTRATIVE EMBODIMENTS

The present inventors have discovered a novel form of therapy of ocular disease that involves vascular targeting of therapeutic agents to ocular neovascular tissue using certain chimeric fusion constructs, which include a vascular endothelial targeting amino acid sequence and a cytotoxic amino acid. This form of therapy can be applied in the treatment and prevention of a wide variety of ophthalmic diseases, such as choroidal neovascularization (CNV) due to any cause including but not limited to age-related macular degeneration, ocular histoplasmosis, pathologic myopia, and angioid streaks. It also applies to retinal neovascularization of any cause including but not limited to proliferative diabetic retinopathy, retinal vein occlusions, and retinopathy of prematurity. It also applies to iris neovascularization and corneal neovascularization of any causes.

### A. POLYPEPTIDES

1. Polypeptides in General

The present invention concerns methods of treating or preventing ocular disease in a subject that involve administration of polypeptides that include a vascular endothelial targeting amino acid sequence and a cytotoxic amino acid sequence. As used herein, a “polypeptide” generally is defined herein to refer to a peptide sequence of about 3 to about 10,000 or more amino acid residues.

The term “amino acid” not only encompasses the 20 common amino acids in naturally synthesized proteins, but also includes any modified, unusual, or synthetic amino acid. One of ordinary skill in the art would be familiar with modified, unusual, or synthetic amino acids. Examples of modified and unusual amino acids are shown on Table 1 below.

TABLE 1
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | β-alanine, | AHyl | allo-Hydroxylysine |
| | β-Amino-propionic acid | | |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, |
| | | | sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

The polypeptides that are included in the methods set forth herein are chimeric in that they comprise a vascular endothelial targeting amino acid sequence and a cytotoxic amino acid sequence. The polypeptides set forth herein may comprise one or more vascular endothelial targeting amino acid sequences, which may or may not be identical. Similarly, the polypeptides set forth herein may comprise one or more cytotoxic amino acid sequences, which may or may not be identical.

In certain embodiments of the present invention, the polypeptide is a fusion polypeptide that includes a vascular endothelial targeting amino acid sequence linked at the N- or C-terminus of the vascular endothelial targeting amino acid sequence to a cytotoxic amino acid sequence. In other embodiments, the polypeptide comprises a linker interposed between the vascular endothelial targeting amino acid sequence and the cytotoxic amino acid sequence. Linkers are discussed in greater detail in the specification below.

Furthermore, the polypeptides set forth herein may comprises a sequence of any number of additional amino acid residues at either the N-terminus or C-terminus of the amino acid sequence that includes the endothelial targeting amino acid sequence and the cytotoxic amino acid sequence. For example, there may be an amino acid sequence of about 3 to about 10,000 or more amino acid residues at either the N-terminus, the C-terminus, or both the N-terminus and C-terminus of the amino acid sequence that includes the endothelial targeting amino acid sequence and the cytotoxic amino acid sequence.

The polypeptide may include the addition of an immunologically active domain, such as an antibody epitope or other tag, to facilitate targeting or purification of the polypeptide. The use of 6×His and GST (glutathione S transferase) as tags is well known. Inclusion of a cleavage site at or near the fusion junction will facilitate removal of the extraneous polypeptide after purification. Other amino acid sequences that may be included in the polypeptide include functional domains, such as active sites from enzymes such as a hydrolase, glycosylation domains, cellular targeting signals or transmembrane regions. The polypeptide may further include one or more additional tissue-targeting moieties, which are discussed in greater detail in the specification below.

Vascular endothelial targeting amino acid sequence and cytotoxic amino acid sequence included in the polypeptides of the present invention may possess deletions and/or substitutions of amino acids relative to the native sequence; thus, sequences with a deletion, sequences with a substitution, and sequences with a deletion and a substitution are contemplated for inclusion in the polypeptides of the present invention. In some embodiments, these targeted polypeptides may further include insertions or added amino acids, such as linkers.

Substitutional or replacement variants typically contain the exchange of one amino acid for another at one or more sites within the protein and may be designed to modulate one or more properties of the polypeptide, particularly to increase its efficacy or specificity. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

In addition to a deletion or substitution, vascular endothelial targeting amino acid sequences and cytotoxic amino acid sequences that are included in the polypeptides set forth herein may possess an insertion one or more residues. This may include the addition of one or more amino acid residues.

The term “biologically functional equivalent” is well understood in the art and is further defined in detail herein. Accordingly, sequences that have between about 70% and about 80%, or between about 81% and about 90%, or even between about 91% and about 99% of amino acids that are identical or functionally equivalent to the amino acids of the native vascular endothelial targeting sequence or cytotic amino acid sequence are included, provided the biological activity of the native sequence is maintained.

Thus, the vascular endothelial targeting amino acid sequences and the cytotoxic amino acid sequences of the polypeptides of the present invention may be biologically functionally equivalent to the native counterparts. For example, the vascular endothelial targeting amino acid sequence may be functionally equivalent in terms of ability to bind or attach to a vascular endothelial cell. In some embodiments, the vascular endothelial targeting amino acid sequence or cytotoxic amino acid sequence may have greater biological activity than their native counterparts.

The following is a discussion based upon changing of the amino acids of a polypeptide to create an equivalent, or even an improved, second-generation molecule. For example, certain amino acids may be substituted for other amino acids in a polypeptide without appreciable loss of function, such as ability to interact with an endothelial cell of a blood vessel. Since it is the interactive capacity and nature of a polypeptide that defines that polypeptide's biological functional activity, certain amino acid substitutions can be made in a polypeptide sequence and nevertheless produce a polypeptide with like properties.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

2. Methods of Polypeptide Synthesis

In certain embodiments of the present invention, the polypeptide is encoded by a single recombinant nucleic acid sequence using recombinant techniques. In other embodiments, the vascular endothelial targeting amino acid sequence and the cytotoxic amino acid sequence have been encoded by separate nucleic acid sequences, and subsequently joined by chemical conjugation. In further embodiments, the polypeptide has been synthesized de novo.

a. Recombinant Techniques In certain embodiments of the present invention, the chimeric polypeptide is encoded by a single recombinant polynucleotide using recombinant techniques well-known to those of ordinary skill in the art. The polynucleotide may include a sequence of additional nucleic acids that direct the expression of the chimeric polypeptide in appropriate host cells.

Due to the inherent degeneracy of the genetic code, other DNA sequences that encode substantially the same or a functionally equivalent amino acid sequence, may be used in the practice of the invention of the cloning and expression of the chimeric protein. Such DNA sequences include those capable of hybridizing to the chimeric sequences or their complementary sequences under stringent conditions. In one embodiment, the phrase “stringent conditions” as used herein refers to those hybridizing conditions that (1) employ low ionic strength and high temperature for washing, for example, 0.015 M NaCl/0.0015 M sodium citrate/0.1% SDS at 50° C.; (2) employ during hybridization a denaturing agent such as formamide, for example, 50% (vol/vol) formamide with a 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.; or (3) employ 50% formamide, 5×SSC (0.75 M NaCl, 0.075 M Sodium pyrophosphate, 5× Denhardt's solution, sonicated salmon sperm DNA (50 μg/ml), 0.1% SDS, and 10% dextran sulfate at 42° C., with washes at 42° C. in 0.2×SSC and 0.1% SDS.

Altered DNA sequences that may be used in accordance with the invention include deletions, additions or substitutions of different nucleotide residues resulting in a sequence that encodes the same or a functionally equivalent polynucleotide. The polynucleotide may contain deletions, additions or substitutions of amino acid residues within a chimeric sequence, which result in a silent change thus producing a functionally equivalent chimeric polynucleotide. Such amino acid substitutions may be made on the basis of similarity in polarity, charge, solubility, hydrophobicity, hydrophilicity, and/or the amphipathic nature of the residues involved, as discussed above.

The DNA sequences of the invention may be engineered in order to alter a chimeric coding sequence for a variety of ends, including but not limited to, alterations that modify processing and expression of the gene product. For example, mutations may be introduced using techniques that are well known in the art, e.g., site-directed mutagenesis, to insert new restriction sites, to alter glycosylation patterns, phosphorylation, etc.

In order to express a biologically active chimeric polypeptide, the nucleotide sequence coding for a chimeric polypeptide, or a functional equivalent, is inserted into an appropriate expression vector, i.e., a vector that contains the necessary elements for the transcription and translation of the inserted coding sequence. The chimeric gene products as well as host cells or cell lines transfected or transformed with recombinant chimeric expression vectors can be used for a variety of purposes. These include, but are not limited to, generating antibodies (i.e., monoclonal or polyclonal) that bind to epitopes of the proteins to facilitate their purification.

Methods that are well known to those skilled in the art can be used to construct expression vectors containing the chimeric coding sequence and appropriate transcriptional/translational control signals. These methods include in vitro recombinant DNA techniques, synthetic techniques and in vivo recombination/genetic recombination. See, for example, the techniques described in Sambrook et al., 2001.

A variety of host-expression vector systems may be utilized to express the chimeric polypeptide coding sequence. These include but are not limited to microorganisms such as bacteria transformed with recombinant bacteriophage DNA, plasmid DNA or cosmid DNA expression vectors containing the chimeric protein coding sequence; yeast transformed with recombinant yeast expression vectors containing the chimeric protein coding sequence; insect cell systems infected with recombinant virus expression vectors (e.g., baculovirus) containing the chimeric protein coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the chimeric protein coding sequence; or animal cell systems. It should be noted that since most apoptosis-inducing proteins cause programmed cell death in mammalian cells, it is preferred that the chimeric protein of the invention be expressed in prokaryotic or lower eukaryotic cells. Section 6 illustrates that IL2-Bax may be efficiently expressed in E. coli.

The expression elements of each system vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter; cytomegalovirus promoter) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll α/β binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5K promoter) may be used; when generating cell lines that contain multiple copies of the chimeric DNA, SV40-, BPV- and EBV-based vectors may be used with an appropriate selectable marker.

In bacterial systems a number of expression vectors may be advantageously selected depending upon the use intended for the chimeric polypeptide expressed. For example, when large quantities of chimeric polypeptide are to be produced, vectors that direct the expression of high levels of protein products that are readily purified may be desirable. Such vectors include but are not limited to the E. coli expression vector pUR278 (Ruther et al., 1983), in which the chimeric protein coding sequence may be ligated into the vector in frame with the lacZ coding region so that a hybrid AS-lacZ protein is produced; pIN vectors (Van Heeke and Schuster, 1989); and the like.

An alternative expression system that could be used to express chimeric polypeptide is an insect system. In one such system, Autographa californica nuclear polyhidrosis virus (AcNPV) is used as a vector to express foreign genes. The virus grows in Spodoptera frugiperda cells. The chimeric protein coding sequence may be cloned into non-essential regions (for example the polyhedrin gene) of the virus and placed under control of an AcNPV promoter (for example the polyhedrin promoter). Successful insertion of the chimeric polypeptide coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (e.g., see Smith et al., 1983; U.S. Pat. No. 4,215,051).

Specific initiation signals may also be required for efficient translation of the inserted chimeric protein coding sequence. These signals include the ATG initiation codon and adjacent sequences. In cases where the entire chimeric gene, including its own initiation codon and adjacent sequences, is inserted into the appropriate expression vector, no additional translational control signals may be needed. However, in cases where the chimeric protein coding sequence does not include its own initiation codon, exogenous translational control signals, including the ATG initiation codon, must be provided. Furthermore, the initiation codon must be in phase with the reading frame of the chimeric protein coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. The presence of consensus N-glycosylation sites in a chimeric protein may require proper modification for optimal chimeric protein function. Different host cells have characteristic and specific mechanisms for the post-translational processing and modification of proteins. Appropriate cell lines or host systems can be chosen to ensure the correct modification and processing of the chimeric protein. To this end, eukaryotic host cells which possess the cellular machinery for proper processing of the primary transcript, glycosylation, and phosphorylation of the chimeric protein may be used. Such mammalian host cells include but are not limited to CHO, VERO, BHK, HeLa, COS, MDCK, 293, W138, and the like.

For long-term, high-yield production of recombinant chimeric polypeptides, stable expression is preferred. For example, cell lines that stably express the chimeric polypeptide may be engineered. Rather than using expression vectors that contain viral originals of replication, host cells can be transformed with a chimeric coding sequence controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines.

A number of selection systems may be used, including but not limited to the herpes simplex virus thymidine kinase (Wigler et al., 1977), hypoxanthine-guanine phosphoribosyltransferase (Szybalski and Szybalski, 1962), and adenine phosphoribosyltransferase (Lowy et al., 1980) genes can be employed in tk-, hgprt- or aprt-cells, respectively. Also, antimetabolite resistance can be used as the basis of selection for dhfr, which confers resistance to methotrexate (Wigler et al., 1980; O'Hare et al., 1981); gpt, which confers resistance to mycophenolic acid (Mulligan and Berg, 1981); neo, which confers resistance to the aminoglycoside G-418 (Colbere-Garapin et al., 1981); and hygro, which confers resistance to hygromycin (Santerre et al., 1984) genes. Additional selectable genes have been described, namely trpB, which allows cells to utilize indole in place of tryptophan; hisD, which allows cells to utilize histinol in place of histidine (Hartman and Mulligan, 1988); and ODC (ornithine decarboxylase) which confers resistance to the ornithine decarboxylase inhibitor, 2-(difluoromethyl)-DL-ornithine, DFMO (see McConlogue, 1986).

b. De novo Synthesis

In an alternate embodiment of the invention, the chimeric polypeptide could be synthesized de novo in whole or in part, using chemical methods well known in the art (see, for example, Caruthers et al., 1980; Crea and Horn, 1980; and Chow and Kempe, 1981). For example, the component amino acid sequences can be synthesized by solid phase techniques, cleaved from the resin, and purified by preparative high performance liquid chromatography followed by chemical linkage to form a chimeric protein. (e.g., see Creighton, 1983). The composition of the synthetic peptides may be confirmed by amino acid analysis or sequencing (e.g., the Edman degradation procedure; see Creighton, 1983).

Polypeptide synthesis techniques are well known to those of skill in the art (see, e.g., Bodanszky et al., 1976). These synthetic methods involve the sequential addition of one or more amino acid residues or suitable protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group, such as lysine.

Using solid phase synthesis as an example, the protected or derivatized amino acid is attached to an inert solid support through its unprotected carboxyl or amino group. The protecting group of the amino or carboxyl group is then selectively removed and the next amino acid in the sequence having the complementary (amino or carboxyl) group suitably protected is admixed and reacted with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to provide the final peptide. Such protecting group moieties may be used in the course of synthesis, but they are removed before the peptides are used. Additional reactions may be necessary, as described elsewhere, to form intramolecular linkages to restrain conformation.

c. Linkers

Alternatively, the two moieties of the chimeric polypeptide produced by synthetic or recombinant methods may be conjugated by linkers according to methods well known in the art (Brinkmann and Pastan, 1994). As used herein, a “linker” is a chemical or peptide or polypeptide that links an endothelial targeting amino acid sequence with a cytotoxic amino acid sequence.

The two coding sequences can be fused directly without any linker or by using a flexible polylinker, such as one composed of the pentamer Gly-Gly-Gly-Gly-Ser (SEQ ID NO:1) repeated 1 to 3 times. Such linker has been used in constructing single chain antibodies (scFv) by being inserted between V_{H} and V_{L} (Bird et al., 1988; Huston et al., 1988). The linker is designed to enable the correct interaction between two beta-sheets forming the variable region of the single chain antibody. Other linkers which may be used include Glu-Gly-Lys-Ser-Ser-Gly-Ser-Gly-Ser-Glu-Ser-Lys-Val-Asp (SEQ ID NO:2) (Chaudhary et al., 1990) and Lys-Glu-Ser-Gly-Ser-Val-Ser-Ser-Glu-Gln-Leu-Ala-Gln-Phe-Arg-Ser-Leu-Asp (SEQ ID NO:3) (Bird et al., 1988).

Multiple peptides or polypeptides may also be joined via a biologically-releasable bond, such as a selectively-cleavable linker or amino acid sequence. For example, peptide linkers that include a cleavage site for an enzyme preferentially located or active within a tumor environment are contemplated. Exemplary forms of such peptide linkers are those that are cleaved by urokinase, plasmin, thrombin, Factor IXa, Factor Xa, or a metallaproteinase, such as collagenase, gelatinase, or stromelysin. Alternatively, polypeptides may be joined to an adjuvant. It can be considered as a general guideline that any linker known to those of ordinary skill in the art is contemplated for use as a linker in the present invention.

It is contemplated that cross-linkers may be implemented with the polypeptide molecules of the present invention. Cross-linking reagents are used to form molecular bridges that tie together functional groups of two different molecules, e.g., a stablizing and coagulating agent. To link two different polypeptides in a step-wise manner, hetero-bifunctional cross-linkers can be used that eliminate unwanted homopolymer formation. Bifunctional cross-linking reagents have been extensively used for a variety of purposes including preparation of affinity matrices, modification and stabilization of diverse structures, identification of binding sites, and structural studies. In the context of the invention, such cross-linker may be used to stabilize the polypeptide or to render it more useful as a therapeutic, for example, by improving the polypeptide's targeting capability or overall efficacy. Cross-linkers may also be cleavable, such as disulfides, acid-sensitive linkers, and others. Homobifunctional reagents that carry two identical functional groups proved to be highly efficient in inducing cross-linking between identical and different macromolecules or subunits of a macromolecule, and linking of polypeptides to specific binding sites on binding partners. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling in one example, of aldehydes to free thiols. The cross-linking reagent can be modified to cross-link various functional groups and is thus useful for cross-linking polypeptides and sugars. In instances where a particular polypeptide, such as gelonin, does not contain a residue amenable for a given cross-linking reagent in its native sequence, conservative genetic or synthetic amino acid changes in the primary sequence can be utilized. Table 2 details certain exemplary hetero-bifunctional cross-linkers considered useful in the present invention.

TABLE 2
HETERO-BIFUNCTIONAL CROSS-LINKERS

| | | | Spacer ArmLength\ | |
|---|---|---|---|---|
| Linker | Reactive Toward | Advantages and Applications | after cross-linking | |
| SMPT | Primary amines | Greater stability | 11.2 | A |
| | Sulfhydryls | | | |
| SPDP | Primary amines | Thiolation | 6.8 | A |
| | Sulfhydryls | Cleavable cross-linking | | |
| LC-SPDP | Primary amines | Extended spacer arm | 15.6 | A |
| | Sulfhydryls | | | |
| Sulfo-LC- | Primary amines | Extended spacer arm | 15.6 | A |
| SPDP | Sulfhydryls | Water-soluble | | |
| SMCC | Primary amines | Stable maleimide reactivegroup | 11.6 | A |
| | Sulfhydryls | Enzyme-antibody conjugation | | |
| | | Hapten-carrier protein conjugation | | |
| Sulfo- | Primary amines | Stable maleimide reactive group | 11.6 | A |
| SMCC | Sulfhydryls | Water-soluble | | |
| | | Enzyme-antibody conjugation | | |
| MBS | Primary amines | Enzyme-antibody conjugation | 9.9 | A |
| | Sulfhydryls | Hapten-carrier protein conjugation | | |
| Sulfo- | Primary amines | Water-soluble | 9.9 | A |
| MBS | Sulfhydryls | | | |
| SIAB | Primary amines | Enzyme-antibody conjugation | 10.6 | A |
| | Sulfhydryls | | | |
| Sulfo- | Primary amines | Water-soluble | 10.6 | A |
| SIAB | Sulfhydryls | | | |
| SMPB | Primary amines | Extended spacer arm | 14.5 | A |
| | Sulfhydryls | Enzyme-antibody conjugation | | |
| Sulfo- | Primary amines | Extended spacer arm | 14.5 | A |
| SMPB | Sulfhydryls | Water-soluble | | |
| EDC/Sulfo- | Primary amines | Hapten-Carrier conjugation | 0 | |
| NHS | Carboxyl groups | | | |
| ABH | Carbohydrates | Reacts with sugar groups | 11.9 | A |
| | Nonselective | | | |

d. Protein Purification

In certain embodiments of the present invention, the polypeptide has been purified. Generally, “purified” will refer to a polypeptide composition that has been subjected to fractionation to remove various other components, and which composition substantially retains its expressed biological activity. Where the term “substantially purified” is used, this designation will refer to a composition in which the polypeptide or peptide forms the major component of the composition, such as constituting about 50% to about 99.9% or more of the proteins in the composition.

Various methods for quantifying the degree of purification of the polypeptide will be known to those of skill in the art in light of the present disclosure. Exemplary techniques include high performance liquid chromatography, ion exchange chromatography, gel electrophoresis, affinity chromatography and the like. The actual conditions used to purify a particular polypeptide will depend, in part, on factors such as net charge, hydrophobicity, hydrophilicity, etc., and will be apparent to those having skill in the art.

For affinity chromatography purification, any antibody that specifically binds the polypeptide may be used. For the production of antibodies, various host animals, including but not limited to rabbits, mice, rats, etc., may be immunized by injection with a chimeric protein or a fragment thereof. The protein may be attached to a suitable carrier, such as bovine serum albumin (BSA), by means of a side chain functional group or linkers attached to a side chain functional group. Various adjuvants may be used to increase the immunological response, depending on the host species, including but not limited to, Freund's (complete and incomplete), mineral gels such as aluminum hydroxide, surface active substances such as lysolecithin, pluronic polyols, polyanions, peptides, oil emulsions, keyhold limpet hemocyanin, dinitrophenol, and potentially useful human adjuvants such as BCG (bacilli Calmetter-Guerin) and Corynebacterium parvum.

Monoclonal antibodies to a chimeric polypeptide may be prepared using any technique that provides for the production of antibody molecules by continuous cell lines in culture. These include but are not limited to the hybridoma technique originally described by Koehler and Milstein (1975), the human B-cell hybridoma technique (Cote et al., 1983), and the EBV-hybridoma technique (Cole et al., 1985). In addition, techniques developed for the production of “chimeric antibodies” (Morrison et al., 1984; Neuberger et al., 1984; Takeda et al., 1985) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce chimeric protein-specific single chain antibodies for chimeric protein purification and detection.

3. Vascular Endothelial Targeting Amino Acid Sequences

The polypeptides set forth in the methods of the present invention comprise at least one vascular endothelial targeting amino acid sequence and at least one cytotoxic amino acid sequence. The therapeutic index of cytotoxic agents can be increased by homing the agents to a site of interest, such as an endothelial cell. A vascular endothelial targeting sequence is defined herein to refer to any amino acid sequence that has the capability to bind (covalently or non-covalently) or otherwise attach to an endothelial cell of a blood vessel. In certain particular embodiments, the blood vessel is a pathological blood vessel, such as a neovascular blood vessel. For example, the neovascular blood vessel may a choroidal neovascular membrane in a patient with age-related macular degeneration, or a frond of retinal neovascularization in a patient with proliferative diabetic retinopathy.

The vascular endothelial cell targeting amino acid sequences that are included in the methods of the present invention may or may not bind to other cell types. Thus, for example, an amino acid sequence that is capable of targeting all cell types, including endothelial cells, in a subject would be a vascular endothelial targeting amino acid sequence since it has the capability to targeting vascular endothelial cells. In certain particular embodiments, the vascular endothelial targeting amino acid sequence is a sequence that preferentially targets endothelial cells relative to other cell types. In certain further particular embodiments of the present invention, the vascular endothelial cell targeting amino acid sequence preferentially binds to neovascular endothelial cells.

The vascular endothelial targeting amino acid sequence confers endothelial cell-specific binding. A wide variety of amino acid sequences are suitable for use as vascular endothelial targeting amino acid sequences, including but not limited to, ligands for receptors such as growth factors, hormones and cytokines, and antibodies or antigen-binding fragments.

Since it is known that tumor cells can form part of the lining of tumor vasculature, the present invention also encompasses targeting to tumor cells directly as well as to the tumor vasculature. Thus, any amino acid sequence that can target tumor cells directly may be applied as vascular endothelial targeting amino acid sequences in the context of the present invention. Many such targeting sequences are known to those of ordinary skill in the art, and these and any which subsequently become available are encompassed within the scope of the present invention.

In certain embodiment, the vascular endothelial targeting amino acid sequence is a binding partner, such as a ligand, of a receptor expressed by an endothelial cell or tumor cell, or a binding partner, such as an antibody, to a marker or a component of the extracellular matrix associated with such cells. More particularly, the targeting sequence may be a binding partner, such as a ligand of a receptor expressed by endothelial cells, or a binding partner, such as an antibody, to an endothelial marker. The term binding partner is used here in its broadest sense and includes both natural and synthetic binding domains, including ligand and antibodies or binding fragments thereof. Thus, the binding partner can be an antibody or a fragment thereof such as Fab, Fv, single-chain Fv, a peptide or a peptido-mimetic, namely a peptido-like molecule capable of binding to the receptor, marker of extracellular component of the cell.

The following are representative examples of vascular endothelial targeting amino acid sequences that can be applied in the context of the present invention. Additional vascular endothelial targeting amino acid sequences known to those of ordinary skill in the art, not specifically set forth herein, are also contemplated for inclusion in the present invention.

a. VEGF

One of the most important growth and survival factors for endothelium is VEGF. VEGF induces angiogenesis and endothelial cell proliferation and it plays an important role in regulating vasculogenesis. VEGF is a heparin-binding glycoprotein that is secreted as a homodimer of 45 kDa. Most types of cells, but usually not endothelial cells themselves, secrete VEGF. In addition, VEGF causes vasodilatation, partly through stimulation of nitric oxide synthase in endothelial cells. VEGF can also stimulate cell migration and inhibit apoptosis. There are several splice variants of VEGF-A. The major ones include: 121, 165, 189 and 206 amino acids (aa), each one comprising a specific exon addition, which are discussed further in other parts of this specification. Exemplary VEGF amino acid sequences suitable for use in the context of the present invention include SEQ ID NOs: 4-10. Additional VEGF amino acid sequences, and chimeric proteins that comprise a VEGF amino acid sequence and a cytotoxic molecule, such as recombinant gelonin, that are useful in the methods of the present invention include those set forth in U.S. Patent Application Pub. No. 20050037967 and U.S. Patent Application Pub. No. 20040248805, each of which is herein specifically incorporated by reference for this section of the specification and all other sections of the specification.

b. EMAP-II

Endothelial-Monocyte Activating Polypeptide-II (EMAP-II) is a cytokine that is an antiangiogenic factor in tumor vascular development, and strongly inhibits tumor growth. Recombinant human EMAP-II is an 18.3 kDa protein containing 166 amino acid residues. EMAP II has also bee found to increase endothelial vessel permeability.

c. PDGF

Platelet-derived growth factor (PDGF) antagonists might increase drug-uptake and therapeutic effects of a broad range of anti-tumor agents in common solid tumors. PDGF is a cytokine of 30 kDA and is released by platelets on wounding and stimulates nearby ells to grow and repair the wound.

d. PD-ECGF

Platelet-derived endothelial cell growth factor (PD-ECGF) was originally isolated from platelets based on its ability to induce mitosis in endothelial cells. Its related protein is gliostatin.

e. Activin

Cells known to express ActRII include endothelial cells. ActRIIB expression parallels that for ActRII, and is again found in endothelial cells. Cells known to express ActRI include vascular endothelial cells. ActRIB has also been identified in endothelial cells.

f. Angiogenin

Angiogenin (ANG) is a 14 kDa, non-glycosylated polypeptide so named for its ability to induce new blood vessel growth.

g. Annexin V

Annexin V is a member of a calcium and phospholipid binding family of proteins with vascular anticoagulant activity. Various synonyms for Annexin V exist: placental protein 4 (PP4), placental anticoagulant protein I (PAP I), calphobindin I (CPB-I), calcium dependent phospholipid binding protein 33 (CaBP33), vascular anticoagulant protein alpha (VACa), anchorin CII, lipocortin-V, endonexin II, and thromboplastin inhibitor. The number of binding sites for Annexin V has been reported as 6-24.times.106/cell in tumor cells and 8.8.times.106/cell for endothelial cells.

h. Ligand of CD44

CD44 is expressed on many cell types. CD44 is remarkable for its ability to generate alternatively spliced forms, many of which differ in their activities. This remarkable flexibility has led to speculation that CD44, via its changing nature, plays a role in some of the methods that tumor cells use to progress successfully through growth and metastasis. CD44 is a 80-250 kDa type I (extracellular N-terminus) transmembrane glycoprotein. Cells known to express CD44H include vascular endothelial cells.

There are multiple ligands for CD44, including osteopontin, fibronectin, collagen types I and IV and hyaluronate. Binding to fibronectin is reported to be limited to CD44 variants expressing chrondroitin sulfate, with the chrondroitin sulfate attachment site localised to exons v8-v11. Hyaluronate binding is suggested to be possible for virtually all CD44 isoforms. One of the principal binding sites is proposed to be centred in exon 2 and to involve lysine and arginine residues. Factors other than the simple expression of a known hyaluronate-binding motif also appear to be necessary for hyaluronate binding. Successful hyaluronate binding is facilitated by the combination of exons expressed, a distinctive cytoplasmic tail, glycosylation patterns, and the activity state of the cell. Thus, in terms of its hyaluronate-binding function, a great deal of “potential” flexibility exists within each CD44-expressing cell.

i. Fibroblast Growth Factor (FGF)

The name “fibroblast growth factor” (FGF) is a limiting description for this family of cytokines. The function of FGFs is not restricted to cell growth. Although some of the FGFs do, indeed, induce fibroblast proliferation, the original FGF molecule (FGF-2 or FGF basic) is now known to also induce proliferation of endothelial cells, chondrocytes, smooth muscle cells, melanocytes, as well as other cells. It can also promote adipocyte differentiation, induce macrophage and fibroblast IL-6 production, stimulate astrocyte migration, and prolong neuronal survival. The FGF superfamily consists of 23 members, all of which contain a conserved 120 amino acid (aa) core region that contains six identical, interspersed amino acids.

Human FGF-1 (also known as FGF acidic, FGFa, ECGF and HBGF-1) is a 17-18 kDa non-glycosylated polypeptide that is expressed by a variety of cells from all three germ layers. The binding molecule may be either an FGF receptor. Cells known to express FGF-1 include endothelial cells.

Human FGF-2, otherwise known as FGF basic, HBGF-2, and EDGF, is an 18 kDa, non-glycosylated polypeptide that shows both intracellular and extracellular activity. Following secretion, FGF-2 is sequestered on either cell surface HS or matrix glycosaminoglycans. Although FGF-2 is secreted as a monomer, cell surface HS seems to dimerize monomeric FGF-2 in a non-covalent side-to-side configuration that is subsequently capable of dimerizing and activating FGF receptors. Cells known to express FGF-2 include endothelial cells.

Human FGF-3 is the product of the int-2 gene. The molecule is synthesized as a 28-32 kDa, 222 aa glycoprotein that contains a number of peptide motifs. Cells reported to express FGF-3 are limited to developmental cells and tumors.

Human FGF-4 is a 22 kDa, 176 aa glycoprotein that is the product of a developmentally-regulated gene. The heparin-binding sites directly relate to FGF4 activity; heparin/heparan regulate the ability of FGF-4 to activate FGFR1 and FGFR2. Cells known to express FGF-4 include both tumor cells and embryonic cells.

j. IL-1R

IL-1 exerts its effects by binding to specific receptors. Two distinct IL-1 receptor binding proteins, plus a non-binding signalling accessory protein have been identified. Each have three extracellular immunoglobulin-like (Ig-like) domains, qualifying them for membership in the type IV cytokine receptor family. The two receptor binding proteins are termed type I IL-1 receptor (IL-1 RI) and type II IL-1 receptor (IL-1 RII) respectively. Human IL-1 RI is a 552 aa, 80 kDa transmembrane glycoprotein that has been isolated from endothelium cells.

k. RTK

The family of receptor tyrosine kinase (RTK), the Eph receptors and their ligands ephrins, have been found to be involved in vascular assembly, angiogenesis, tumorigenesis, and metastasis. It has also been that class A Eph receptors and their ligands are elevated in tumor and associated vasculature.

l. MMP

Matrix metalloproteinases (MMPs) have been implicated in tumor growth, angiogenesis, invasion, and metastasis.

m. NG2

NG2 is a large, integral membrane, chondroitin sulfate proteoglycan that was first identified as a cell surface molecule expressed by immature neural cells. It is expressed by a wide variety of immature cells as well as several types of tumors with high malignancy. NG2 has been suggested as a target molecule in tumor vasculature.

n. Oncofetal Fibronectin

The expression of the oncofetal fragment of fibronectin (Fn-f) has also been found to be increased during angiogenesis. It has been suggested as a marker of tumor angiogenesis.

o. Ligand of Tenascin

Tenascin is a matrix glycoprotein seen in malignant tumors including brain and breast cancers and melanoma. Its expression in malignant but not well-differentiated tumors and association with the blood vessels of tumors makes it an important target for malignant tumors and angiogenesis. The targeting moiety is preferably a polypeptide that is capable of binding to a tumor cell or tumor vasculature surface molecule.

p. TNF

TNF acts as an inflammatory cytokine and has the effect of inducing alteration of the endothelial barrier function, reducing of tumor interstitial pressure, and increasing chemotherapeutic drug penetration and tumor vessel damage. TNF-related ligands usually share a number of common features. These features do not include a high degree of overall amino acid (aa) sequence homology. With the exception of nerve growth factor (NGF) and TNF-beta, all ligands are synthesized as type II transmembrane proteins (extracellular C-terminus) that contain a short cytoplasmic segment (10-80 aa residues) and a relatively long extracellular region (140-215 aa residues).

q. PDGF

PDGF binds to receptors that are expressed in the stromal compartment in most common solid tumors. Inhibition of stromally expressed PDGF receptors in a rat colon carcinoma model reduces the tumor interstitial fluid pressure and increases tumor transcapillary transport.

r. Ligands of CAMS and Selectins

Cell adhesion molecules (CAMs) are cell surface proteins involved in the binding of cells, usually leukocytes, to each other, to endothelial cells, or to extracellular matrix. Specific signals produced in response to wounding and infection control the expression and activation of certain of these adhesion molecules. The interactions and responses then initiated by binding of these CAMs to their receptors/ligands play important roles in the mediation of the inflammatory and immune reactions that constitute one line of defense against these insults. Most of the CAMs characterized so far fall into three general families of proteins: the immunoglobulin (Ig) superfamily, the integrin family, or the selectin family.

A member of the Selectin family of cell surface molecules, L-Selectin consists of an NH2-terminal lectin type C domain, an EGF-like domain, two complement control domains, a 15 amino acid residue spacer, a transmembrane sequence and a short cytoplasmic domain.

Three ligands for L-Selectin on endothelial cells have been identified, all containing O-glycosylated mucin or mucin-like domains. P-Selectin, a member of the Selectin family of cell surface molecules, consists of an NH2-terminal lectin type C domain, an EGF-like domain, nine complement control domains, a transmembrane domain, and a short cytoplasmic domain.

The tetrasacharide sialyl Lewisx (sLex) has been identified as a ligand for both P-Selectin and E-Selectin, but P- E- and L-Selectin can all bind sLex and sLea under appropriate conditions. P-Selectin also reportedly binds selectively to a 160 kDa glycoprotein present on murine myeloid cells and to a glycoprotein on myeloid cells, blood neutrophils, monocytes, and lymphocytes termed P-Selectin glycoprotein ligand-1 (PSGL-1), a ligand that also can bind E-Selectin. P-Selectin-mediated rolling of leukocytes can be completely inhibited by a monoclonal antibody specific for PSLG-1, suggesting that even though P-Selectin can bind to a variety of glycoproteins under in vitro conditions, it is likely that physiologically important binding is more limited. A variety of evidence indicates that P-Selectin is involved in the adhesion of myeloid cells, as well as B and a subset of T cells, to activated endothelium.

s. Ligands of Ig Superfamily CAMs

The Ig superfamily CAMs are calcium-independent transmembrane glycoproteins. Members of the Ig superfamily include the intercellular adhesion molecules (ICAMs), vascular-cell adhesion molecule (VCAM-1), platelet-endothelial-cell adhesion molecule (PECAM-1), and neural-ceu adhesion molecule (NCAM). Each Ig superfamily CAM has an extracellular domain, which contains several Ig-like intrachain disulfide-bonded loops with conserved cysteine residues, a transmembrane domain, and an intracellular domain that interacts with the cytoskeleton. Typically, they bind integrins or other Ig superfamily CAMs. The neuronal CAMs have been implicated in neuronal patterning. Endothelial CAMs play an important role in immune response and inflammation.

Human CD31 is a 130 kDa, type I (extracellular N-terminus) transmembrane glycoprotein that belongs to the cell adhesion molecule (CAM) or C2-like subgroup of the IgSF1. The mature molecule is 711 amino acid (aa) residues in length and contains a 574 aa residue extracellular region, a 19 aa residue transmembrane segment, and a 118 aa residue cytoplasmic tail. In the extracellular region, there are nine potential N-linked glycosylation sites, and, with a predicted molecular weight of 80 kDa, it appears many of these sites are occupied. The most striking feature of the extracellular region is the presence of six Ig-homology units that resemble the C2 domains of the IgSF. Although they vary in number, the presence of these modules is a common feature of all IgSF adhesion molecules (ICAM-1, 2, 3 & VCAM-1).

t. Ligands of Integrins

Integrins are non-covalently linked heterodimers of alpha and beta subunits. Numerous alpha and beta subunits have been identified. These can combine in various ways to form different types of integrin receptors. The ligands for several of the integrins are adhesive extracellular matrix (ECM) proteins such as fibronectin, vitronectin, collagens and laminin. Many integrins recognize the amino acid sequence RGD (arginine-glycine-aspartic acid) which is present in fibronectin or the other adhesive proteins to which they bind. Peptides and protein fragments containing the RGD sequence can be used to modulate the activities of the RGD-recognising integrins. Thus the present invention may employ as the targeting moiety peptides recognized by integrins. These peptides are conventionally known as “RGD-containing peptides”. These peptides may include peptides motifs that have been identified as binding to integrins. These motifs include the amino acid sequences: DGR, NGR and CRGDC. The peptide motifs may be linear or cyclic. One of ordinary skill in the art would be familiar with these amino acid sequences.

u. Cytokines

Exemplary cytokines include IL-1, IL-1, IL-6, IFN-alpha., INF-beta, IL-11, INF-gamma, TGF-beta., IL-8, PF-4, PBP, NAP-2, beta.-TG, MIP-1 alpha., MIP-1 beta., MCP-1, MCP-2, MCP-3, RANTES C, IL-12 LIF, OSM, CNTF, PF-4, PBP, NAP-2, beta.-TG, MIP, and MCP.

v. Chemokines

Chemokines are a superfamily of mostly small, secreted proteins that play a critical role in many pathophysiological processes, including angiogenesis. There are at least seventeen known chemokine receptors, and many of these receptors exhibit promiscuous binding properties whereby several different chemokines can signal through the same receptor. Chemokines are divided into subfamilies based on conserved aa sequence motifs. Most family members have at least four conserved cysteine residues that form two intramolecular disulfide bonds. The subfamilies are defined by the position of the first two cysteine residues.

w. Antibody

In certain embodiments, the vascular endothelial targeting amino acid sequence is an antibody sequence, such as an antibody sequence directed against an antigen expressed on the surface of an endothelial cell. The endothelial cell targeting sequence may be derived from heavy and/or light chain sequences from an immunoglobulin (Ig) variable region. Such a variable region may be derived from a natural human antibody or an antibody from another species such as a rodent antibody. Alternatively the variable region may be derived from an engineered antibody such as a humanized antibody or from a phage display library from an immunised or a non-immunized animal or a mutagenized phage-display library. As a second alternative, the variable region may be derived from a single-chain variable fragment (scFv). The polypeptide may contain other sequences to achieve multimerization or to act as spacers between the component domains of the polypeptide. The targeting sequence may comprise, in addition to one or more immunoglobulin variable regions, all or part of an Ig heavy chain constant region and so may comprise a natural whole Ig, an engineered Ig, an engineered Ig-like molecule, a single-chain Ig or a single-chain Ig-like molecule.

As used herein, an “antibody” refers to a protein consisting of one or more polypeptides substantially encoded by immunoglobulin genes or fragments of immunoglobulin genes. Antibodies may exist as intact immunoglobulins or as a number of fragments, including those well-characterised fragments produced by digestion with various peptidases. While various antibody fragments are defined in terms of the digestion of an intact antibody, one of skill will appreciate that antibody fragments may be synthesised de novo either chemically or by utilising recombinant DNA methodology. Thus, the term antibody, as used herein also includes antibody fragments either produced by the modification of whole antibodies or synthesised de novo using recombinant DNA methodologies. Antibody fragments encompassed by the use of the term “antibodies” include, but are not limited to, Fab, Fab′, F (ab′) 2, scFv, Fv, dsFv diabody, and Fd fragments.

The invention also contemplates monoclonal or polyclonal antibodies directed against endothelial cell surface proteins. Thus, the present invention further provides a process for the production of monoclonal or polyclonal antibodies to endothelial cell surface proteins.

If polyclonal antibodies are desired, a selected mammal (e.g., mouse, rabbit, goat, horse, etc.) is immunized with an immunogenic polypeptide bearing an epitope(s). Serum from the immunized animal is collected and treated according to known procedures. If serum containing polyclonal antibodies to an epitope contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenized to another polypeptide for use as immunogens in animals or humans.

Monoclonal antibodies directed against binding cell surface epitopes in the polypeptides can also be readily produced by one skilled in the art. The general methodology for making monoclonal antibodies by hybridomas is well known in the art. Immortal antibody-producing cell lines can be created by cell fusion, and also by other techniques such as direct transformation of B lymphocytes with oncogenic DNA, or transfection with Epstein-Barr virus. Panels of monoclonal antibodies produced against epitopes can be screened for various properties; i.e., for isotype and epitope affinity.

x. Other

Other examples of vascular targeting amino acid sequences include other ligands that bind to tumor cells, such as those set forth in U.S. Patent App. Pub. No. 20030040496, which is herein incorporated by reference in its entirety. Additional examples of vascular endothelial targeting amino acid sequences COX-2 inhibitors, anti-EGF receptor ligands, herceptin, angiostatin, C225, and thalidomide. COX-2 inhibitors include, for example, celecoxib, rofecoxib, etoricoxib, and analogs of these agents. Other examples of vascular endothelial targeting amino acid sequences include mda-7 and related amino acid sequences (discussed in greater detail in U.S. patent application Ser. No. 10/791,692, herein specifically incorporated by reference in its entirety).

4. Cytotoxic Amino Acid Sequences

A cytotoxic amino acid sequence is defined herein to refer to any amino acid sequence that is capable of causing injury or death to a cell.

a. Ribosome-Inhibitory Toxins

In certain particular embodiments of the methods set forth herein, the cytotoxic amino acid sequence is a ribosome-inhibitory toxin (RIT). RITs are potent inhibitors of protein synthesis in eukaryotes. The enzymatic domain of these proteins acts as a cytotoxic n-glycosidase that is able to inactivate catalytically ribosomes once they gain entry to the intracellular compartment. This is accomplished by cleaving the n-glycosidic bond of the adenine at position 4324 in the 28srRNA, which irreversibly inactivates the ribosome apparently by disrupting the binding site for elongation factors. RITs, which have been isolated from bacteria, are prevalent in higher plants. In plants, there are two types: Type I toxins possess a single polypeptide chain that has ribosome inhibiting activity, and Type II toxins have an A chain, comparable to the Type I protein, that is linked by a disulfide bond to a B chain possessing cell-binding properties. Examples of Type I RITs are gelonin, dodecandrin, tricosanthin, tricokirin, bryodin, mirabilis antiviral protein, barley ribosome-inactivating protein (BRIP), pokeweed antiviral proteins (PAPs), saporins, luffins, and momordins. Type II toxins include ricin and abrin. Toxins may be conjugated or expressed as a fusion protein with any of the polypeptides discussed herein.

b. Other Toxins

Any toxin known to those of ordinary skill in the art is suitable for the polypeptides of the present invention. Exemplary toxins include ricin A-chain (Burbage, 1997), diphtheria toxin A (Massuda et al., 1997; Lidor, 1997), pertussis toxin A subunit, E. coli enterotoxin toxin A subunit, cholera toxin A subunit and Pseudomonas toxin c-terminal are suitable. It has demonstrated that transfection of a plasmid containing the fusion protein regulatable diphtheria toxin A chain gene was cytotoxic for cancer cells. Other exemplary toxins envisioned as useful for the present invention include Abrin, A/B heat labile toxins, Botulinum toxin, Helix pomatia, Jacalin or Jackfruit, Peanut agglutinin, Sambucus nigra, Tetanus, Ulex, and Viscumin.

C. Pro-Apoptotic Amino Acid Sequences

Pro-apoptotic amino acid sequences including sequences that induce or sustain apoptosis to an active form. The present invention contemplates inclusion of any pro-apoptotic amino acid sequence known to those of ordinary skill in the art. Exemplary pro-apoptotic amino acid sequences include CD95, caspase-3, Bax, Bag-1, CRADD, TSSC3, bax, hid, Bak, MKP-7, PERP, bad, bc1-2, MST1, bbc3, Sax, BIK, BID, and mda7. One of ordinary skill in the art would be familiar with pro-apoptotic amino acid sequences, and other such sequences not specifically set forth herein that can be applied in the methods and compositions of the present invention.

d. Anti-Angiogenic Amino Acid Sequences

Other examples of cytotoxic amino acid sequences include anti-angiogenic amino acid sequences. An anti-angiogenic amino acid sequence is defined herein to refer to any amino acid sequence that inhibits the development of or promotes regression of angiogenesis. To the extent that these sequences can be used to target vascular endothelial cells, some of these sequences can also function as vascular endothelial targeting amino acid sequences in the context of the present invention.

Examples of anti-angiogenic amino acid sequences are set forth in U.S. Patent App. Pub. No. 20030082159 and U.S. Patent App. Pub. No. 20020114783, each of which is herein incorporated by reference in their entirety for this section of the specification and all other sections of the specification.

Any anti-angiogenic amino acid sequence known to those of ordinary skill in the art is contemplated for inclusion in the present invention. Examples of such sequences include, but are not limited to the following:

(1) Tissue Inhibitors of Metalloproteinases

The tissue inhibitors of metalloproteinases (TIMPs) represent a family of ubiquitous proteins that are natural inhibitors of the matrix metalloproteinases (MMPs). Matrix metalloproteinases are a group of zinc-binding endopeptidases involved in connective tissue matrix remodeling and degradation of the extracellular matrix (ECM), an essential step in tumor invasion, angiogenesis, and metastasis. The matrix metalloproteinases each have different substrate specificities within the extracellular matrix and are important in its degradation. The analysis of matrix metalloproteinases in human mammary pathology showed that several matrix metalloproteinases were involved in degradation of the extracellular matrix: collagenase (MMP1) degrades fibrillar interstitial collagens; gelatinase (MMP2) mainly degrades type IV collagen; and stromelysin (MMP3) has a wider range of action.

There are four members of the TIMP family. TIMP-1 and TIMP-2 are capable of inhibiting tumor growth, invasion, and metastasis that has been related to matrix metalloproteinase inhibitory activity. Furthermore, both TIMP-1 and TIMP-2 are involved in the inhibition of angiogenesis. Unlike other members of the TIMP family, TIMP-3 is found only in the ECM and may function as a marker for terminal differentiation. Finally, TIMP-4 is thought to function in a tissue-specific fashion in extracellular matrix hemostasis (Gomez et al., 1997).

Tissue inhibitor of metalloproteinase-1 (TIMP-1) is a 23 kD protein that is also known as metalloproteinase inhibitor 1, fibroblast collagenase inhibitor, collagenase inhibitor and erythroid potentiating activity (EPA). The gene encoding TIMP-1 has been described by Docherty et al. (1985). TIMP-1 complexes with metalloproteinases (such as collagenases) and causes irreversible inactivation. The effects of TIMP-1 have been investigated in transgenic mouse models: one that overexpressed TIMP-1 in the liver, and another that expressed the viral oncogene Simian Virus 40/T antigen (TAg) leading to heritable development of hepatocellular carcinomas. In double transgenic experiments in which the TIMP-1 lines were crossed with the TAg transgenic line, overexpression of hepatic TIMP-1 was reported to block the development of TAg-induced hepatocellular carcinomas by inhibiting growth and angiogenesis (Martin et al., 1996).

Tissue inhibitor of metalloproteinase-2 (TIMP-2) is a 24 kD protein that is also known as metalloproteinase inhibitor 2. The gene encoding TIMP-2 has been described by Stetler-Stevenson et al. (1990). Metalloproteinase (MMP2) which plays a critical role in tumor invasion is complexed and inhibited by TIMP-2. Thus, TIMP-2 could be useful to inhibit cancer metastasis (Musso et al., 1997). When B16F10 murine melanoma cells, a highly invasive and metastatic cell line, were transfected with a plasmid coding for human TIMP-2 and injected subcutaneously in mice, TIMP-2 over-expression limited tumor growth and neoangiogenesis in vivo (Valente et al., 1998).

Tissue inhibitor of metalloproteinase-3 (TIMP-3) is also known as metalloproteinase inhibitor 3. When breast carcinoma and malignant melanoma cell lines were transfected with TIMP-3 plasmids and injected subcutaneously into nude mice, suppression of tumor growth was observed (Anand-Apte et al., 1996). However, TIMP-3 over-expression had no effect on the growth of the two tumor cell lines in vitro. Thus, it was suggested that the TIMP-3 released to the adjacent extracellular matrix by tumor cells inhibited tumor growth by suppressing the release of growth factors sequestered in extracellular matrix, or by inhibiting angiogenesis (Anand-Apte et al., 1996).

Tissue inhibitor of metalloproteinase-4 (TIMP-4) is also known as metalloproteinase inhibitor 4. The TIMP-4 gene and tissue localization have been described by Greene et al. (1996). Biochemical studies have shown that TIMP-4 binds human gelatinase A similar to that of TIMP-2 (Bigg et al., 1997). The effect of TIMP-4 modulation on the growth of human breast cancers in vivo was investigated by Wang et al. (1997). Overexpression of TIMP-4 was found to inhibit cell invasiveness in vitro, and tumor growth was significantly reduced following injection of nude mice with TIMP-4 tumor cell transfectants in vivo (Wang et al., 1997).

(2) Endostatin, Angiostatin, PEX, Kringle-5

Boehm et al. (1997) showed that treatment of mice with Lewis lung carcinomas with the combination of endostatin+angiostatin proteins induced complete regression of the tumors, and that mice remained healthy for the rest of their life. This effect was obtained only after one cycle (25 days) of endostatin+angiostatin treatment, whereas endostatin alone required 6 cycles to induce tumor dormancy.

Bergers et al. (1999) demonstrated a superior antitumoral effect of the combination of endostatin+angiostatin proteins in a mouse model for pancreatic islet carcinoma. Endostatin+angiostatin combination resulted in a significant regression of the tumors, whereas endostatin or angiostatin alone had no effect.

(3) Endostatin XVIII

Endostatin, an angiogenesis inhibitor produced by hemangioendothelioma, was first identified by O'Reilly et al. (1997). Endostatin is a 20 kD C-terminal fragment of collagen XVIII that specifically inhibits endothelial proliferation, and potently inhibits angiogenesis and tumor growth. In fact, primary tumors have been shown to regress to dormant microscopic lesions following the administration of recombinant endostatin (O'Reilly et al., 1997). Endostatin is reported to inhibit angiogenesis by binding to the heparin sulfate proteoglycans involved in growth factor signaling (Zetter, 1998).

(4) Endostatin XV

Recently, a C-terminal fragment of collagen XV (Endostatin XV) has been shown to inhibit angiogenesis like Endostatin XVIII, but with several functional differences (Sasaki et al., 2000).

(5) Angiostatin

Angiostatin, an internal fragment of plasminogen comprising the first four kringle structures, is one of the most potent endogenous angiogenesis inhibitors described to date. It has been shown that systemic administration of angiostatin efficiently suppresses malignant glioma growth in vivo (Kirsch et al., 1998). Angiostatin has also been combined with conventional radiotherapy resulting in increased tumor eradication without increasing toxic effects in vivo (Mauceri et al., 1998). Other studies have demonstrated that retroviral and adenoviral mediated gene transfer of angiostatin cDNA resulted in inhibition of endothelial cell growth in vitro and angiogenesis in vivo. The inhibition of tumor-induced angiogenesis produced an increase in tumor cell death (Tanaka et al., 1998). Gene transfer of a cDNA coding for mouse angiostatin into murine T241 fibrosarcoma cells has been shown to suppress primary and metastatic tumor growth in vivo (Cao et al., 1998).

(6) PEX

PEX is the C-terminal hemopexin domain of MMP-2 that inhibits the binding of MMP-2 to integrin αvβ₃ and blocks cell surface collagenolytic activity required for angiogenesis and tumor growth. It was cloned and described by Brooks et al. (1998).

(7) Kringle-5

The kringle-5 domain of human plasminogen, which shares high sequence homology with the four kringles of angiostatin, has been shown to be a specific inhibitor for endothelial cell proliferation. Kringle-5 appears to be more potent than angiostatin on inhibition of basic fibroblast growth factor-stimulated capillary endothelial cell proliferation (Cao et al., 1997). In addition to its antiproliferative properties, kringle-5 also displays an anti-migratory activity similar to that of angiostatin that selectively affects endothelial cells (Ji et al., 1998).

(8) Chemokines

Chemokines are low-molecular weight pro-inflammatory cytokines capable of eliciting leukocyte chemotaxis. Depending on the chemokine considered, the chemoattraction is specific for certain leukocyte cell types. Moreover, in addition to their chemotactic activity, some chemokines possess an anti-angiogenic activity, i.e. they inhibit the formation of blood vessels feeding the tumor. For this reason, these chemokines are useful in cancer treatment.

(9) Monokine-Induced By Interferon-Gamma (MIG)

MIG, the monokine-induced by interferon-gamma, is a CXC chemokine related to IP-10 and produced by monocytes. MIG is a chemoattractant for activated T cells, and also possesses strong angiostatic properties. Intratumoral injections of MIG induced tumor necrosis (Sgadari et al., 1997).

(10) Interferon-Alpha Inducible Protein 10 (IP-10)

IP-10, the interferon-alpha inducible protein 10, is a member of the CXC chemokine family. IP-10 is produced mainly by monocytes, but also by T cells, fibroblasts and endothelial cells. IP-10 exerts a chemotactic activity on lymphoid cells such as T cells, monocytes and NK cells. IP-10 is also a potent inhibitor of angiogenesis. It inhibits neovascularization by suppressing endothelial cell differentiation. Because of its chemotactic activity toward immune cells, IP-10 was considered as a good candidate to enhance antitumour immune responses. Gene transfer of IP-10 into tumor cells reduced their tumorigenicity and elicited a long-term protective immune response (Luster and Leder, 1993). The angiostatic activity of IP-10 was also shown to mediate tumor regression. Tumor cells expressing IP-10 became necrotic in vivo (Sgadari et al., 1996). IP-10 was also shown to mediate the angiostatic effects of IL-12 that lead to tumor regression (Tannenbaum et al., 1998).

(11) VEGF Receptors

FLT-1 (fms-like tyrosine kinase 1 receptor) is a membrane-bound receptor of VEGF (VEGF Receptor 1). It has been shown that a soluble fragment of FLT-1 (sFLT-1) has angiostatic properties by way of its antagonist activity against VEGF. Soluble FLT-1 acts by binding to VEGF but also because it binds and blocks the external domain of the membrane-bound FLT-1. One example of sFLT-1 is a human sFLT-1 spanning the 7 immunoglobulin-like domains of the external part of FLT-1.

(12) sFLK-1/KDR

FLK-1 or KDR (kinase insert domain receptor) is a membrane-bound receptor of VEGF (VEGF Receptor 2). It has been shown that a soluble fragment of KDR (sKDR) has angiostatic properties by way of its antagonist activity against VEGF. The sKDR also binds and blocks the external domain of the membrane-bound KDR. One example of sKDR is a human sKDR spanning the 7 immunoglobulin-like domains of the external part of KDR.

5. Other Tissue-Targeting Moieties

In certain embodiments of the present invention, the polypeptide includes one or more additional tissue targeting moieties other than the vascular endothelial cell targeting amino acid sequence. The one or more additional tissue targeting moieties may either be recombinantly expressed or chemically conjugated to the polypeptide comprising the vascular endothelial cell targeting amino acid sequence and the cytotoxic amino acid sequence.

A “tissue-targeting moiety” is defined herein to refer to a part of a molecule that can bind or attach to tissue. The binding may be by any mechanism of binding known to those of ordinary skill in the art. Examples include antimetabolites, apoptotic agents, bioreductive agents, signal transductive therapeutic agents, receptor responsive agents, or cell cycle specific agents. The tissue may be any type of tissue, such as a cell. For example, the cell may be the cell of a subject, such as a cancer cell or a cell type that is specific to the eye.

In some embodiments the tissue-targeting moiety is a “targeting ligand.” A “targeting ligand” is defined herein to be a molecule or part of a molecule that binds with specificity to another molecule. One of ordinary skill in the art would be familiar with the numerous agents that can be employed as targeting ligands in the context of the present invention.

Examples of targeting ligands include disease cell cycle targeting compounds, tumor angiogenesis targeting ligands, tumor apoptosis targeting ligands, disease receptor targeting ligands, drug-based ligands, antimicrobials, tumor hypoxia targeting ligands, an agent that mimics glucose, amifostine, angiostatin, EGF receptor ligands, capecitabine, COX-2 inhibitors, deoxycytidine, fullerene, herceptin, human serum albumin, lactose, leuteinizing hormone, pyridoxal, quinazoline, thalidomide, transferrin, and trimethyl lysine.

In further embodiments of the present invention, the tissue-targeting moiety is an antibody. Any antibody is contemplated as a tissue-targeting moiety in the context of the present invention. For example, the antibody may be a monoclonal antibody. One of ordinary skill in the art would be familiar with monoclonal antibodies, methods of preparation of monoclonal antibodies, and methods of use of monoclonal antibodies as ligands. In certain embodiments of the present invention, the monoclonal antibody is an antibody directed against a tumor marker. In some embodiments, the monoclonal antibody is monoclonal antibody C225, monoclonal antibody CD31, or monoclonal antibody CD40.

A single tissue-targeting moiety, or more than one such tissue-targeting moiety, may be bound to a polypeptide of the present invention. In these embodiments, any number of tissue-targeting moieties may be bound to the polypeptides set forth herein. Thus, there may be one or more tissue targeting moieties attached to a polypeptide of the present invention. The tissue-targeting moieties can be bound to the polypeptide in any manner. For example, the tissue-targeting moiety may be bound to the polypeptide in an amide linkage, or in an ester linkage. One of ordinary skill in the art would be familiar with the chemistry of these agents, and methods to incorporate these agents as moieties of the polypeptides of the claimed invention. Methods of synthesis of the compounds of the present invention are discussed in detail below.

Information pertaining to tissue targeting moieties and conjugation of these moieties to polypeptides are provided in U.S. Pat. No. 6,692,724, U.S. patent application Ser. No. 09/599,152, U.S. patent application Ser. No. 10/627,763, U.S. patent application Ser. No. 10/672,142, U.S. patent application Ser. No. 10/703,405, U.S. patent application Ser. No. 10/732,919, each of which is herein specifically incorporated by reference.

### B. EYE DISEASE

The present invention pertains to methods of treating or preventing eye disease in a subject that involve administering to the subject a therapeutically effective amount of a pharmaceutical composition comprising a polypeptide having a vascular endothelial targeting amino acid sequence and a cytotoxic amino acid sequence. The subject can be any subject, such as a mammal. Mammals include, but not limited to, humans, horses, dogs, and cats. The subject may also be a non-mammal, such as an avian species. In certain particular embodiments, the subject is a human.

Any eye disease that can afflict a subject is contemplated by the present invention. In certain particular embodiments, the eye disease is a disease associated with neovascularization. For example, the neovascularization may be corneal neovascularization, retinal neovascularization, choroidal neovascularization, or iris neovascularization.

Exemplary diseases contemplated for prevention or treatment by the methods of the present invention include age-related macular degeneration associated with choroidal neovascularization, proliferative diabetic retinopathy (diabetic retinopathy associated with retinal, preretinal, or iris neovascularization), proliferative vitreoretinopathy, retinopathy of prematurity, or a condition associated with ischemia, such as branch retinal vein occlusion, central retinal vein occlusion, branch retinal artery occlusion, or central retinal artery occlusion. The eye disease may also be an infectious eye disease, such as HIV retinopathy, toxocariasis, toxoplasmosis, endophthalmitis, and so forth. Any eye disease associated with inflammation is also contemplated for treatment using the methods of the present invention. Exemplary inflammatory eye diseases include, but are not limited to, uveitis, endophthalmitis, ophthalmic trauma, ophthalmic surgery, and so forth.

In other embodiments, the neovascularization is tumor neovascularization. The tumor may be either a benign or malignant tumor. Exemplary benign tumors include hamartomas and neurofibromas. Exemplary malignant tumors include choroidal melanoma, uveal melanoma or the iris, uveal melanoma of the ciliary body, retinoblastoma, or metastatic disease (e.g., choroidal metastasis).

The neovascularization may also be neovascularization associated with an ocular wound. For example, the wound may the result of a traumatic injury to the globe, such as a corneal laceration. Alternatively, the wound may be the result of ophthalmic surgery. In some embodiments, the methods of the present invention may be applied to prevent or reduce the risk of proliferative vitreoretinopathy following vitreoretinal surgery, prevent corneal haze following corneal surgery (such as corneal transplantation and eximer laser surgery), prevent closure of a trabeculectomy, prevent or substantially slow the recurrence of pterygii, and so forth.

The neovascularization may be located either on or within the eye of the subject. For example, the neovascularization may be corneal neovascularization (either located on the corneal epithelium or on the endothelial surface of the cornea), iris neovascularization, neovascularization within the vitreous cavity, retinal neovasculization, or choroidal neovascularization. The neovascularization may also be neovascularization associated with conjunctival disease.

The subject may not only be a subject with an active eye disease, but may be a subject at risk of developing an eye disease. For example, the subject may have a genetic predisposition for developing a particular eye disease. Alternatively, the subject may be a subject with an eye disease that affects one eye, but not the fellow eye.

### C. PHARMACEUTICAL COMPOSITIONS AND THERAPEUTICS

1. Verification of Functional Activity

Once a chimeric polypeptide is synthesized, its identity and functional activities can be readily determined by methods well known in the art. For example, antibodies to the two amino acid sequences of the chimeric polypeptide may be used to identify the protein in Western blot analysis. In addition, the chimeric polypeptide can be tested for specific binding to target cells in binding assays using a fluorescent-labeled or radiolabelled secondary antibody.

2. Therapeutically Effective Amount

Pharmaceutical compositions of the present invention comprise a therapeutically effective amount of a chimeric polypeptides as set forth herein. The phrases “pharmaceutical composition” refers to compositions that do not produce an unacceptable adverse, allergic or other untoward reaction when administered to a subject, such as, for example, a human. The specificity of VEGF-gelonin fusion constructs has been demonstrated. See, e.g., U.S. Patent Publication nos. 20050037967 and 20040248805, incorporated herein specifically incorporated by reference in its entirety.

Through delivery of the compositions of the present invention, unwanted growth of cells may be slowed or halted, thus ameliorating or preventing the disease. The methods utilized herein specifically target and kill or halt proliferation of vascular endothelial cells. This treatment is suitable for warm-blooded animals: mammals, including, but not limited to, humans, horses, dogs, and cats, and for non-mammals, such as avian species.

A therapeutically effective amount of one or more of the chimeric polypeptides set forth herein is administered to subject for the treatment or prevention of eye disease in a subject. The term “therapeutically effective amount” is defined herein as the amount of a chimeric polypeptide of the present invention that treats, prevents, eliminates, decreases, delays, or minimizes adverse effects of an eye disease. Eye diseases contemplated for treatment are discussed in greater detail in the specification above. A skilled artisan readily recognizes that in many cases the chimeric polypeptide may not provide a cure but may only provide partial benefit. In some embodiments, a physiological change having some benefit is also considered therapeutically beneficial. Thus, in some embodiments, an amount of chimeric polypeptide that provides a physiological change is considered an “effective amount” or a “therapeutically effective amount.” A therapeutically effective amount may be administered as a single dosage or may be administered according to a regimen involving more than one dose over a period of time. Repeated administration may be required to achieve the desired effect.

Therapeutically effective concentrations and amounts may be determined empirically by testing the chimeric polypeptide in known in vitro and in vivo systems, such as those described here; dosages for humans or other animals may then be extrapolated therefrom.

The preparation of therapeutically effective or diagnostically effective compositions will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

3. Formulations

Pharmaceutical compositions set forth herein may be manufactured by means of conventional mixing, dissolving, granulating, dragee-making, levigating, emulsifying, encapsulating, entrapping or lyophilizing processes. Pharmaceutical compositions may be formulated in any manner known to those of ordinary skill in the art. They may be formulated in any manner using one or more physiologically acceptable carriers, diluents, excipients or auxiliaries which facilitate processing of the proteins into preparations which can be used pharmaceutically. Proper formulation is dependent upon the route of administration chosen.

Pharmaceutical carriers or vehicles suitable for administration of the compositions provided herein include any such carriers known to those skilled in the art to be suitable for the particular mode of administration. Exemplary carriers include solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. Except insofar as any conventional carrier is incompatible with the active ingredient, its use in the present compositions is contemplated.

In some embodiments, the composition comprises various antioxidants to retard oxidation of one or more component. Additionally, the prevention of the action of microorganisms can be brought about by preservatives such as various antibacterial and antifungal agents, including, but not limited to parabens (e.g., methylparabens, propylparabens), chlorobutanol, phenol, sorbic acid, thimerosal or combinations thereof.

The therapeutic compositions of the present invention may be formulated in a free base, free acid, neutral or salt form. Pharmaceutically acceptable salts include the salts formed with the free carboxyl groups derived from inorganic bases such as for example, sodium, potassium, ammonium, calcium or ferric hydroxides; or such organic bases as isopropylamine, trimethylamine, histidine or procaine.

A carrier can be a solvent or dispersion medium comprising, but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

Solutions or suspensions used for parenteral, intraocular, intravitreal, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of toxicity such as sodium chloride or dextrose. Parental preparations can be enclosed in ampules, disposable syringes or multiple dose vials made of glass, plastic or other suitable material.

If administered intravenously or intraocularly, suitable carriers include physiological saline or phosphate buffered saline (PBS), and solutions containing thickening and solubilizing agents, such as glucose, polyethylene glycol, and polypropylene glycol and mixtures thereof.

Sterile injectable solutions are prepared by incorporating the therapeutic agent in the required amount of the appropriate solvent with various amounts of the other ingredients enumerated above, as required, followed by filtered sterilization. Generally, dispersions are prepared by incorporating the various sterilized active ingredients into a sterile vehicle that contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

In some embodiments, the active agent is formulated as a solid or powder to be combined with a vehicle. Upon mixing or addition of the active agent with the vehicle, the resulting mixture may be a solution, suspension, emulsion or the like. The form of the resulting mixture depends upon a number of factors, including the intended mode of administration and the solubility of the conjugate in the selected carrier or vehicle. The effective concentration is sufficient for ameliorating the symptoms of the disease, disorder or condition treated and may be empirically determined based upon in vitro and/or in vivo data, such as the data from the mouse xenograft model for tumors or rabbit ophthalmic model. If necessary, pharmaceutically acceptable salts or other derivatives of the conjugates and complexes may be prepared.

The active materials can also be mixed with other active materials, that do not impair the desired action, or with materials that supplement the desired action, including viscoelastic materials, such as hyaluronic acid, which is sold under the trademark HEALON (solution of a high molecular weight (MW of about 3 millions) fraction of sodium hyaluronate; manufactured by Pharmacia, Inc. see, e.g. U.S. Pat. No. 5,292,362, U.S. Pat. No. 5,282,851, U.S. Pat. No. 5,273,056, U.S. Pat. No. 5,229,127, U.S. Pat. No. 4,517,295 and U.S. Pat. No. 4,328,803), VISCOAT (fluorine-containing (meth)acrylates, such as, 1H,1H,2H,2H-hepta-decafluo-rodecylmethacrylate; see, e.g., U.S. Pat. No. 5,278,126, U.S. Pat. No. 5,273,751 and U.S. Pat. No. 5,214,080; commercially available from Alcon Surgical, Inc.), ORCOLON (see, e.g., U.S. Pat. No. 5,273,056; commercially available from Optical Radiation Corporation), methylcellulose, methyl hyaluronate, polyacrylamide and polymethacrylamide (see, e.g., U.S. Pat. No. 5,273,751). The viscoelastic materials are present generally in amounts ranging from about 0.5 to 5.0%, preferably 1 to 3% by weight of the conjugate material and serve to coat and protect the treated tissues. The compositions may also include a dye, such as methylene blue or other inert dye, so that the composition can be seen when injected into the eye or contacted with the surgical site during surgery.

The therapeutic agents of the present invention may be prepared with carriers that protect them against rapid degradation or elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others. For example, the composition may be applied during surgery using a sponge, such as a commercially available surgical sponges (see, e.g., U.S. Pat. No. 3,956,044 and U.S. Pat. No. 4,045,238; available from Weck, Alcon, and Mentor), that has been soaked in the composition and that releases the composition upon contact with the eye. These are particularly useful for application to the eye for ophthalmic indications following or during surgery in which only a single administration is possible. The compositions may also be applied in pellets (such as Elvax pellets (ethylene-vinyl acetate copolymer resin); about 1-5.mu.g of conjugate per 1 mg resin) that can be implanted in the eye during surgery.

Preferably, the chimeric polypeptides to be administered are substantially pure. As used herein, “substantially pure” means sufficiently homogeneous to appear free of readily detectable impurities as determined by standard methods of analysis, such as thin layer chromatography (TLC), gel electrophoresis, high performance liquid chromatography (HPLC), used by those of skill in the art to assess such purity, or sufficiently pure such that further purification would not detectably alter the physical and chemical properties, such as enzymatic and biological activities, of the substance. Methods for purification of the polypeptides to produce substantially chemically pure polypeptides are known to those of skill in the art, and are addressed in greater detail above. A substantially chemically pure compound may, however, be a mixture of stereoisomers. In such instances, further purification might increase the specific activity of the polypeptide.

The therapeutic agents of the present invention can be administered by any method known to those of ordinary skill in the art. The therapeutic agent may be formulated into pharmaceutical compositions suitable for topical, local, intravenous and systemic application. In certain particular embodiments, the therapeutic agent is administered intraocularly. For example, injection can be intravitreal, transeptal, subconjunctival, or into the anterior chamber. Other routes of administration include periocular administration and topical administration. In certain other embodiments, injection can be intravenously, intradermally, intraarterially, intralesionally, intracranially, topically, intratumorally, subcutaneously, subconjunctivally, submucosally, intranasally, or orally. Time release formulations are also desirable.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

Alternatively, other pharmaceutical delivery systems may be employed. Liposomes and emulsions are well known examples of delivery vehicles that may be used to deliver the therapeutic polypeptides. These are discussed in greater detail below in this specification. Certain organic solvents such as dimethylsulfoxide also may be employed, although usually at the cost of greater toxicity. Additionally, the proteins may be delivered using a sustained-release system, such as semipermeable matrices of solid polymers containing the therapeutic agent. Various of sustained-release materials have been established and are well known by those skilled in the art. Sustained-release capsules may, depending on their chemical nature, release the proteins for a few weeks up to over 100 days. Depending on the chemical nature and the biological stability of the chimeric protein, additional strategies for protein stabilization may be employed.

As the proteins of the invention may contain charged side chains or termini, they may be included in any of the above-described formulations as the free acids or bases or as pharmaceutically acceptable salts. Pharmaceutically acceptable salts are those salts which substantially retain the biologic activity of the free bases and which are prepared by reaction with inorganic acids. Pharmaceutical salts tend to be more soluble in aqueous and other protic solvents than are the corresponding free base forms.

The chimeric polypeptides may also be formulated for local or topical application, such as for topical application to the skin and mucous membranes, such as in the eye, in the form of drops, gels, creams, and lotions. Such solutions may be formulated in any manner known to those of ordinary skill in the art. For example, they may be formulated as 0.01%-10% isotonic solutions, pH about 5-7, with appropriate salts. The ophthalmic compositions may also include additional components, such as hyaluronic acid. The conjugates and complexes may be formulated as aerosols for topical application (see, U.S. Pat. No. 4,044,126, U.S. Pat. No. 4,414,209, and U.S. Pat. No. 4,364,923, each of which is herein specifically incorporated by reference).

The therapeutic agent can also be mixed with other active agents that do not impair the desired action, or with other agents that supplement the desired action, such as another therapeutic agent directed against neovascularization. Finally, the therapeutic agent may be packaged as articles of manufacture containing packaging material, one or more therapeutic agents as provided herein within the packaging material, and a label detailing the indication for which the therapeutic agent is provided.

4. Effective Dosages

The actual required amount of a composition of the present invention administered to a subject can be determined by physical and physiological factors such as body weight, severity of condition, the type of disease being treated or prevented, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of a chimeric polypeptide. In other embodiments, the polypeptide may comprise between about 2% to about 75% of the weight of the unit, or between about 25% to about 60%, for example, and any range derivable therein. In other non-limiting examples, a dose may also comprise from about 0.1 mg/kg/body weight to about 1000 mg/kg/body weight or any amount within this range, or any amount greater than 1000 mg/kg/body weight per administration Additional information regarding dosage of VEGF/gelonin fusion constructs can be found in U.S. Patent Publication nos. 20050037967 and 20040248805, herein specifically incorporated by reference in its entirety.

The chimeric polypeptides set forth herein will generally be used in an amount effective to achieve the intended purpose. For use to treat or prevent a disease condition, the proteins of the invention, or pharmaceutical compositions thereof, are administered or applied in a therapeutically effective amount. A therapeutically effective amount is an amount effective to ameliorate or prevent the symptoms, or prolong the survival of, the patient being treated. Determination of a therapeutically effective amount is well within the capabilities of those skilled in the art, especially in light of the detailed disclosure provided herein.

For systemic administration, a therapeutically effective dose can be estimated initially from in vitro assays. Initial dosages can also be estimated from in vivo data, e.g., animal models, using techniques that are well known in the art. One having ordinary skill in the art could readily optimize administration to humans based on animal data.

### D. COMBINATION TREATMENTS/CANCER THERAPIES

In order to increase the effectiveness of a chimeric polypeptide of the present invention, or expression construct coding therefor, it may be desirable to combine these compositions with other agents effective in the treatment of eye disease. For example, the chimeric polypeptides of the present invention can be administered in conjunction with other treatment of ocular neovascularization. If the neovascularization is secondary to age-related macular degeneration, for example, the other treatment may be laser photocoagulation, nutritional therapy, or any other form of therapy for age-related macular degeneration. If the eye disease is cancer, such as ocular melanoma, metastasis tumor, or retinoblastoma, for example, then the other form of therapy may be an anti-cancer therapy.

An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell.

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that chimeric polypeptides could be used similarly in conjunction with chemotherapeutic, radiotherapeutic, gene therapy, or immunotherapeutic intervention, in addition to other pro-apoptotic or cell cycle regulating agents.

Alternatively, the therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, chimeric polypeptide therapy is “A” and the secondary agent is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the therapeutic chimeric polypeptides of the present invention to a subject will follow general protocols for the administration of therapeutic agents, taking into account the toxicity, if any, of the chimeric polynucleotide. It is expected that the treatment cycles would be repeated as necessary. It also is contemplated that various standard therapies may be applied in combination with the described chimeric polypeptides.

1. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabine, navelbine, farnesyl-protein transferase inhibitors, transplatinum, 5-fluorouracil, vincristine, vinblastin and methotrexate, or any analog or derivative variant of the foregoing.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

3. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

4. Genes

In yet another embodiment, the secondary treatment is a gene therapy in which a therapeutic polynucleotide is administered before, after, or at the same time as a chimeric polypeptide of the present invention. Delivery of a chimeric polypeptide in conjunction with a second vector encoding one of the following gene products will have a combined anti-hyperproliferative effect on target tissues. Examples include tumor suppressor genes and pro-apoptotic genes.

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

6. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### E. LIPID COMPOSITIONS

In certain embodiments, the present invention employs a novel composition comprising one or more lipids associated with at least one chimeric polypeptide. A lipid is a substance that is characteristically insoluble in water and extractable with an organic solvent. Lipids include, for example, the substances comprising the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes. Of course, compounds other than those specifically described herein that are understood by one of skill in the art as lipids are also encompassed by the compositions and methods of the present invention.

A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof.

1. Lipid Types

A neutral fat may comprise a glycerol and a fatty acid. A typical glycerol is a three carbon alcohol. A fatty acid generally is a molecule comprising a carbon chain with an acidic moeity (e.g., carboxylic acid) at an end of the chain. The carbon chain may of a fatty acid may be of any length, however, it is preferred that the length of the carbon chain be of from about 2 to about 30 or more carbon atoms, and any range derivable therein. However, a preferred range is from about 14 to about 24 carbon atoms in the chain portion of the fatty acid, with about 16 to about 18 carbon atoms being particularly preferred in certain embodiments. In certain embodiments the fatty acid carbon chain may comprise an odd number of carbon atoms, however, an even number of carbon atoms in the chain may be preferred in certain embodiments. A fatty acid comprising only single bonds in its carbon chain is called saturated, while a fatty acid comprising at least one double bond in its chain is called unsaturated.

Specific fatty acids include, but are not limited to, linoleic acid, oleic acid, palmitic acid, linolenic acid, stearic acid, lauric acid, myristic acid, arachidic acid, palmitoleic acid, arachidonic acid ricinoleic acid, tuberculosteric acid, lactobacillic acid. An acidic group of one or more fatty acids is covalently bonded to one or more hydroxyl groups of a glycerol. Thus, a monoglyceride comprises a glycerol and one fatty acid, a diglyceride comprises a glycerol and two fatty acids, and a triglyceride comprises a glycerol and three fatty acids.

A phospholipid generally comprises either glycerol or an sphingosine moiety, an ionic phosphate group to produce an amphipathic compound, and one or more fatty acids. Types of phospholipids include, for example, phophoglycerides, wherein a phosphate group is linked to the first carbon of glycerol of a diglyceride, and sphingophospholipids (e.g., sphingomyelin), wherein a phosphate group is esterified to a sphingosine amino alcohol. Another example of a sphingophospholipid is a sulfatide, which comprises an ionic sulfate group that makes the molecule amphipathic. A phopholipid may, of course, comprise further chemical groups, such as for example, an alcohol attached to the phosphate group. Examples of such alcohol groups include serine, ethanolamine, choline, glycerol and inositol. Thus, specific phosphoglycerides include a phosphatidyl serine, a phosphatidyl ethanolamine, a phosphatidyl choline, a phosphatidyl glycerol or a phosphotidyl inositol. Other phospholipids include a phosphatidic acid or a diacetyl phosphate. In one aspect, a phosphatidylcholine comprises a dioleoylphosphatidylcholine (a.k.a. cardiolipin), an egg phosphatidylcholine, a dipalmitoyl phosphalidycholine, a monomyristoyl phosphatidylcholine, a monopalmitoyl phosphatidylcholine, a monostearoyl phosphatidylcholine, a monooleoyl phosphatidylcholine, a dibutroyl phosphatidylcholine, a divaleroyl phosphatidylcholine, a dicaproyl phosphatidylcholine, a diheptanoyl phosphatidylcholine, a dicapryloyl phosphatidylcholine or a distearoyl phosphatidylcholine.

A glycolipid is related to a sphinogophospholipid, but comprises a carbohydrate group rather than a phosphate group attached to a primary hydroxyl group of the sphingosine. A type of glycolipid called a cerebroside comprises one sugar group (e.g., a glucose or galactose) attached to the primary hydroxyl group. Another example of a glycolipid is a ganglioside (e.g., a monosialoganglioside, a GM1), which comprises about 2, about 3, about 4, about 5, about 6, to about 7 or so sugar groups, that may be in a branched chain, attached to the primary hydroxyl group. In other embodiments, the glycolipid is a ceramide (e.g., lactosylceramide).

A steroid is a four-membered ring system derivative of a phenanthrene. Steroids often possess regulatory functions in cells, tissues and organisms, and include, for example, hormones and related compounds in the progestagen (e.g., progesterone), glucocorticoid (e.g., cortisol), mineralocorticoid (e.g., aldosterone), androgen (e.g., testosterone) and estrogen (e.g., estrone) families. Cholesterol is another example of a steroid, and generally serves structural rather than regulatory functions. Vitamin D is another example of a sterol, and is involved in calcium absorption from the intestine.

A terpene is a lipid comprising one or more five carbon isoprene groups. Terpenes have various biological functions, and include, for example, vitamin A, coenzyme Q and carotenoids (e.g., lycopene and β-carotene).

2. Charged and Neutral Lipid Compositions

In certain embodiments, a lipid component of a composition is uncharged or primarily uncharged. In one embodiment, a lipid component of a composition comprises one or more neutral lipids. In another aspect, a lipid component of a composition may be substantially free of anionic and cationic lipids, such as certain phospholipids (e.g., phosphatidyl choline) and cholesterol. In certain aspects, a lipid component of an uncharged or primarily uncharged lipid composition comprises about 95%, about 96%, about 97%, about 98%, about 99% or 100% lipids without a charge, substantially uncharged lipid(s), and/or a lipid mixture with equal numbers of positive and negative charges.

In other aspects, a lipid composition may be charged. For example, charged phospholipids may be used for preparing a lipid composition according to the present invention and can carry a net positive charge or a net negative charge. In a non-limiting example, diacetyl phosphate can be employed to confer a negative charge on the lipid composition, and stearylamine can be used to confer a positive charge on the lipid composition.

Lipids can be obtained from natural sources, commercial sources or chemically synthesized, as would be known to one of ordinary skill in the art. For example, phospholipids can be from natural sources, such as egg or soybean phosphatidylcholine, brain phosphatidic acid, brain or plant phosphatidylinositol, heart cardiolipin and plant or bacterial phosphatidylethanolamine.

3. Lipid Composition Structures

In some embodiments of the present invention, the chimeric polypeptide is associated with a lipid. A chimeric polypeptide associated with a lipid may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure. A lipid or lipid/chimeric polypeptide associated composition of the present invention is not limited to any particular structure. For example, they may also simply be interspersed in a solution, possibly forming aggregates which are not uniform in either size or shape. In another example, they may be present in a bilayer structure, as micelles, or with a “collapsed” structure. In another non-limiting example, a lipofectamine (Gibco BRL)-chimeric polypeptide or Superfect (Qiagen)-chimeric polypeptide complex is also contemplated.

A lipid composition may comprise about 1% to about 100%, or any range derivable therein, of a particular lipid, lipid type or non-lipid component such as a drug, protein, sugar, nucleic acids or other material disclosed herein or as would be known to one of skill in the art. A lipid may be comprised in an emulsion. A lipid emulsion is a substantially permanent heterogenous liquid mixture of two or more liquids that do not normally dissolve in each other, by mechanical agitation or by small amounts of additional substances known as emulsifiers. Methods for preparing lipid emulsions and adding additional components are well known in the art (e.g., Modern Pharmaceutics, 1990, incorporated herein by reference). A lipid may be comprised in a micelle. A micelle is a cluster or aggregate of lipid compounds, generally in the form of a lipid monolayer, and may be prepared using any micelle producing protocol known to those of skill in the art (e.g., Canfield et al., 1990; El-Gorab et al, 1973; Colloidal Surfactant, 1963; and Catalysis in Micellar and Macromolecular Systems, 1975, each incorporated herein by reference). In particular embodiments, a lipid comprises a liposome. A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition. One of ordinary skill in the art would be familiar with liposomes, and delivery of therapeutic agents using liposomes.

### F. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Vascular Targeting of Ocular Neovascularization with a VEGF₁₂₁/Gelonin Chimeric Protein

In this study, the effects of systemic or intraocular administration of a VEGF₁₂₁/gelonin chimeric protein (VEGF/rGel) on several animal models of ocular neovascularization were tested. VEGF/rGel has previously been shown to cause infarction of tumor vessels (Veenendaal et al., 2002).

Materials

The fusion toxin, VEGF/rGel, and recombinant gelonin (rGel) were expressed in bacterial cultures, purified to homogeneity and characterized for biological activity as previously described (Veenendaal et al., 2002). The fusion toxin and rGel were stored in PBS at −20° C.

Model of choroidal neovascularization. Mice were treated in accordance with the Association for Research in Vision and Ophthalmologoy (ARVO) Statement for the Use of Animals in Ophthalmic and Vision Research and the U.S. National Institutes of Health Guide for the Care and Use of Laboratory Animals. The model of laser-induced choroidal neovascularization has been previously described (Tobe et al., 1998). Briefly, 4- to 6-week-old female C57BL/6J mice were anesthetized with ketamine hydrochloride (100 mg/kg body weight) and pupils were dilated with 1% tropicamide. A 532 nm diode laser photocoagulator (OcuLight GL; Iridex, Mountain View, Calif.) with a slit lamp delivery system was used with a cover slip as a contact lens to visualize the retina and deliver sufficient laser energy (75 μm spot size, 0.1 seconds duration, 140 mW) to rupture Bruch's membrane in 3 locations in each eye, the 9, 12, and 3 o'clock positions of the posterior pole. Production of a bubble at the time of laser burn, which indicates rupture of Bruch's membrane, is an important factor in obtaining experimental CNV (Tobe et al., 1998); therefore, only burns in which a bubble was produced were included in the study.

One week after rupture of Bruch's membrane, 7 mice were anesthetized and perfused with fluorescein-labeled dextran (2×10⁶ average mw, Sigma, St. Louis, Mo.) and the amount of choroidal neovascularization at Bruch's membrane rupture sites was measured on choroidal flat mounts. Other mice received experimental or control injections one week after rupture of Bruch's membrane and choroidal neovascularization was assessed one week later. To test the effect of intravenous administration of VEGF/rGel, mice were given tail vein injections (every 2 days for a total of 4 injections) of 45 mg/kg of VEGF/rGel (8 mice) or rGel (8 mice), or PBS vehicle alone (7 mice). One week later, they were perfused with fluorescein-labeled dextran and choroidal neovascularization was measured on choroidal flat mounts. To test the effect of intravitreous administration of VEGF/rGel, 9 mice were given an intravitreous injection of 5 ng of rGel in one eye and PBS in the fellow eye and 13 mice were given 5 ng of VEGF/rGel in one eye and PBS in the fellow eye. After one week, mice were perfused with fluorescein-labeled dextran and choroidal neovascularization was measured on choroidal flat mounts.

Rho/VEGF Transgenic Mice. Transgenic mice in which the rhodopsin promoter drives expression of VEGF in photoreceptors (rho/VEGF mice) have been previously described (Okamoto et al., 1997; Tobe et al., 1998). Hemizygous rho/VEGF (line V6) transgenics in a C57BL/6 background were used for all experiments. At P21, the baseline amount of subretinal neovascularization was measured in eight mice. Nine mice received intravitreous injections at P21; 5 ng of VEGF/rGel was injected in one eye and 5 ng of rGel was injected in the other eye. At P25, the amount of subretinal neovascularization was measured in each eye.

Quantification of Neovascularization on Flat Mounts. In mice with laser-induced choroidal neovascularization, the neovascularization was measured on choroidal flat mounts and in rho/VEGF transgenics, subretinal neovascularization was measured on retinal flat mounts. Flat mounts were prepared as previously described (Tobe et al., 1998; Nambu et al., 2003). After mice were terminally perfused with fluorescein-labeled dextran, eyes were removed, fixed for 1 hour in 10% phosphate-buffered formalin, and the cornea and lens were removed. The entire retina was carefully dissected from the eyecup, and depending upon the model, the retina or choroid was flat mounted in Aquamount after 4 radial cuts were made in each quadrant. Flat mounts were examined by fluorescence microscopy using an Axioskop microscope (Zeiss, Thormwood, N.Y.) and images were digitized using a 3 CCD color video camera (IK-TU40A, Toshiba, Tokyo, Japan) and a frame grabber. Retinas were mounted with photoreceptor side up and examined with 400× magnification, which provides a narrow depth of field so that when focusing on the outer edge of the retina the retinal vessels are out-of-focus in the background allowing easy delineation of the subretinal neovascularization. Image-Pro Plus software (Media Cybernetics, Silver Spring, Md.) was used to measure the area of each subretinal or choroidal neovascularization lesion.

Immunofluorescent localization of VEGF/rGel. One week after rupture of Bruch's membrane, mice were given 2.5 mg/kg of VEGF/rGel or rGel, or vehicle alone by tail vein injection. Forty-five minutes later, mice were given an intraperitoneal injection of 300 U of heparin and 15 minutes later, mice were terminally perfused by pumping saline into the left ventricle at 1 ml/minute for 12 minutes. Eyes were removed and frozen in optimum cutting temperature embedding compound (OCT; Miles Diagnostics, Elkhart, Ind.). Frozen sections were cut and adjacent sections were stained with biotinylated Griffonia simplicifolia lectin B4 (GSA), which selectively stains vascular cells, or 10 μg/ml of rabbit anti-rGel antibody (Veenendaal et al., 2002). Rabbit anti-rGel antibody was detected with goat anti-rabbit IgG conjugated to FITC (Jackson ImmunoResearch, West Grove, Pa.).

Histochemical Staining with GSA Lectin. Slides were incubated in methanol/H₂O₂ for 10 minutes at 4° C., washed with 0.05 M TBS and incubated for 30 minutes in 10% normal porcine serum. Slides were incubated 2 hours at room temperature with biotinylated GSA lectin (Vector Laboratories, Burlingame, Calif.) and after rinsing with 0.05 M TBS, they were incubated with avidin coupled to peroxidase (Vector Laboratories) for 45 minutes at room temperature. After a 10-minute wash in 0.05 M TBS, slides were incubated with diaminobenzidine (Research Genetics, Huntsville, Ala.) to produce a brown reaction product.

Mice with Oxygen-Induced Ischemic Retinopathy. Ischemic retinopathy was produced by a previously described method (Smith et al., 1994). At P7, litters of mice were placed in an airtight incubator and exposed to an atmosphere of 75±3% oxygen for 5 days. Incubator temperature was maintained at 23±2° C., and oxygen was measured every 8 hours with an oxygen analyzer. After 5 days, the mice were removed from the incubator and placed in room air. At P17, six mice were euthanized to measure the baseline amount of neovascularization and seven mice were given an intravitreous injection of 5 ng of VEGF/rGel in one eye and 5 ng of rGel in the other eye. At P21, mice were euthanized to measure retinal neovascularization.

Measurement of Retinal Neovascularization in Mice with Ischemic Retinopathy. After euthanasia, eyes were rapidly removed and frozen in OCT. Ocular frozen sections (10 μm) were histochemically stained with GSA, as described above. Slides were counterstained with eosin, which stains the internal limiting membrane, and mounted (Cytoseal; Stephens Scientific, Cornwall, N.J.). To perform quantitative assessments, 10 μm serial sections were cut through the entire eye starting with sections that included the iris root on one side of the eye and proceeding to the iris root on the other side. Every tenth section, roughly 100 μm apart, was stained with GSA and images were digitized with a three CCD color video camera and a frame grabber. Image analysis was used to delineate GSA-stained cells on the surface of the retina, and their area was measured. The mean area of neovascularization per section was calculated for each eye and was used as a single experimental value.

Statistical Analyses. Statistical comparisons were made using a linear mixed model (Verbeke and Molenberghs, 2000). This model is analogous to analysis of variance (ANOVA), but allows analysis of all choroidal neovascularization area measurements from each mouse rather than average choroidal neovascularization area per mouse by accounting for correlation between measurements from the same mouse. The advantage of this model over ANOVA is that it accounts for differing precision in mouse-specific average measurements arising from a varying number of observations among mice. P-values for comparison of treatments were adjusted for multiple comparisons using Bonferroni/Dunn's method.

Results

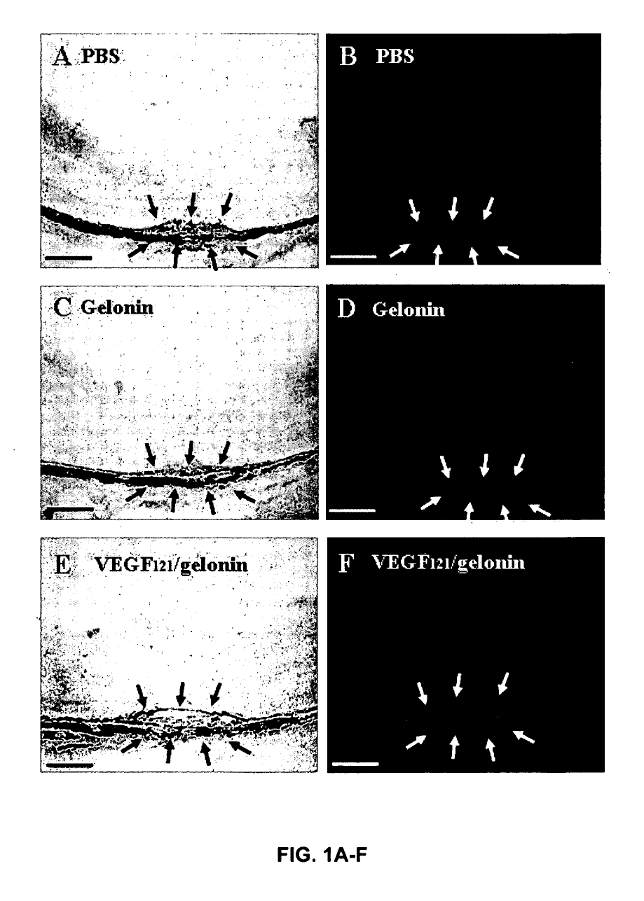

VEGF/rGel localizes to choroidal neovascularization after intravenous injection. Seven days after laser-induced rupture of Bruch's membrane, mice were given a tail vein injection of PBS, rGel, or VEGF/rGel and after 1 hour the mice were euthanized and ocular sections were histochemically stained with GSA or immunofluorescently stained with anti-gelonin antibody. Mice that had received an injection of PBS or rGel showed choroidal neovascularization at Bruch's membrane rupture sites (FIG. 1A and FIG. 1C, arrows) that did not stain for gelonin (FIG. 1B and FIG. 1D, arrows). In contrast, mice that had received an intravenous injection of VEGF/rGel, showed staining for gelonin within choroidal neovascularization (FIG. 1E-F, arrows).

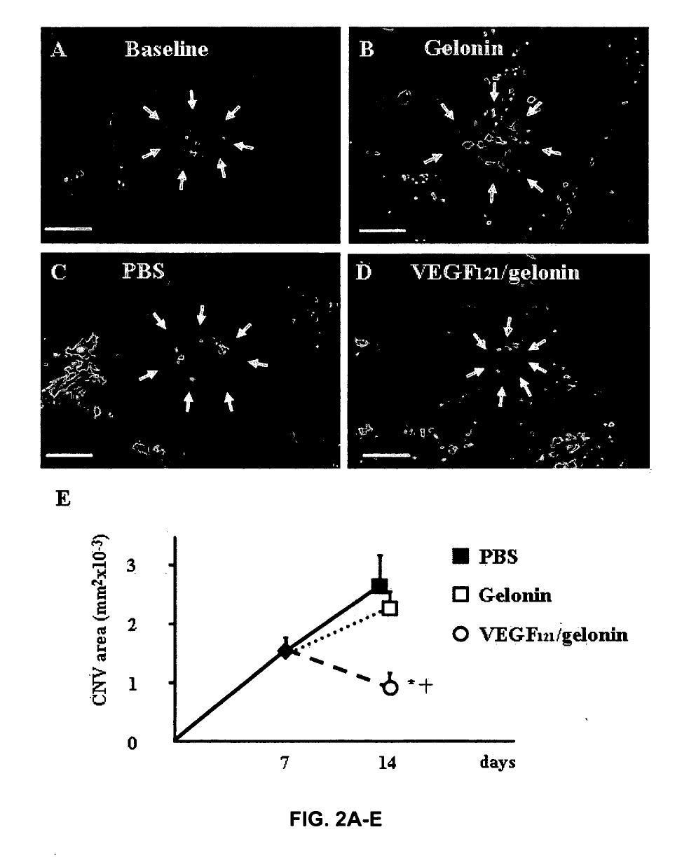

Intravenous injection of VEGF/rGel causes regression of choroidal neovascularization. Forty adult C57BL/6 mice had laser-induced rupture of Bruch's membrane in three locations in each eye. After 1 week, 7 mice were perfused with fluorescein-labeled dextran and the baseline amount of choroidal neovascularization at rupture sites (FIG. 2A, arrows) was measured by image analysis of choroidal flat mounts. The remaining mice received a tail vein injection of 45 mg/kg of rGel, 45 mg/kg of VEGF/rGel, or PBS. One week after the intravenous injection, the mice were perfused with fluorescein-labeled dextran and choroidal flat mounts were examined by fluorescence microscopy. The area of choroidal neovascularization (mm²×10⁻³) at rupture sites appeared smaller in mice that had been injected with VEGF/rGel (0.95±0.20, FIG. 2D, arrows) compared to those in mice that had been injected with rGel (2.25±0.30, FIG. 2B, arrows) or PBS (2.65±0.48, FIG. 2C, arrows), and a statistically significant difference was confirmed by image analysis (FIG. 2E). They were also smaller than baseline choroidal neovascularization lesions present on day 7 (1.56±0.14, FIG. 2A and FIG. 2E), indicating that VEGF/rGel caused regression of choroidal neovascularization.

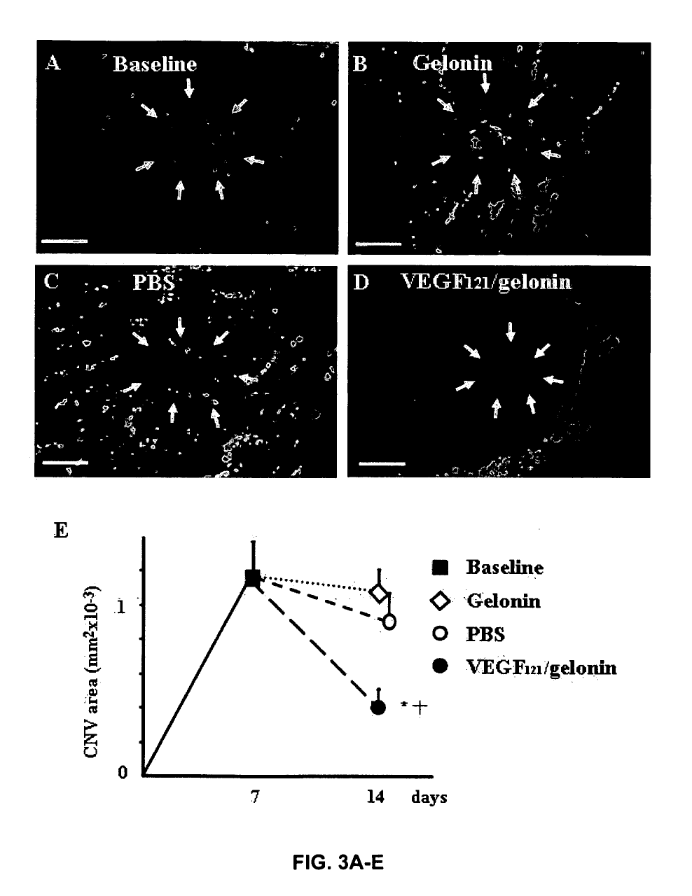

Intravitreous injection of VEGF/rGel causes regression of choroidal neovascularization. Thirty-one adult C57BL/6 mice had laser-induced rupture of Bruch's membrane in three locations in each eye. After 1 week, 9 mice were perfused with fluorescein-labeled dextran, choroidal flat mounts were prepared, and the baseline amount of choroidal neovascularization at rupture sites (FIG. 3A) was measured. The remaining mice were divided into 2 groups; 9 mice received an intravitreous injection of 5 ng of rGel in one eye and PBS in the fellow eye and 13 mice received 5 ng of VEGF/rGel in one eye and PBS in the fellow eye. After 1 week, the mice were perfused with fluorescein-labeled dextran and choroidal flat mounts were examined by fluorescence microscopy. The area of choroidal neovascularization at rupture sites was less in mice that had been injected with VEGF/rGel (0.43±0.07; FIG. 3D and FIG. 3E, arrows) compared to that in mice that had been injected with rGel (1.03±0.17; FIG. 3B and FIG. 3E, arrows) or PBS (0.92±0.20; FIG. 3C, arrows). It was also smaller than the amount of choroidal neovascularization seen at baseline (1.19±0.19; FIG. 3A and FIG. 3E).

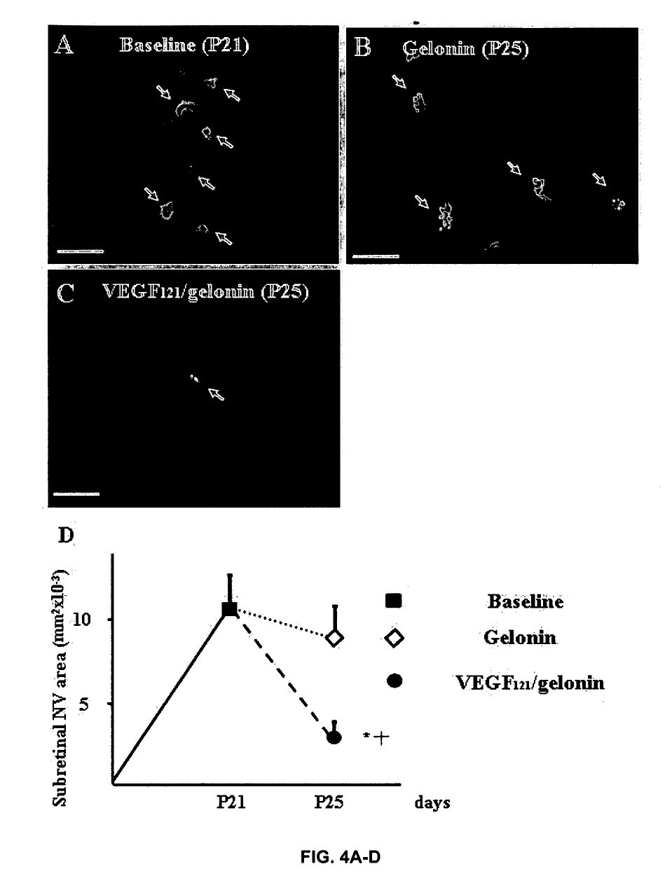

Intravitreous injection of VEGF/rGel causes regression of neovascularization in rho/VEGF transgenic mice. Transgenic mice in which the rhodopsin promoter drives expression of VEGF in photoreceptors (rho/VEGF mice) develop subretinal neovascularization that is quite consistent among mice of the same line in the same genetic background and is easily quantified by image analysis of retinal flat mounts after perfusion of mice with fluorescein-labeled dextran (Okamoto et al., 1997; Tobe et al., 1998). Eight hemizygous rho/VEGF mice in a C57BL/6 background had the baseline amount of neovascularization per retina (mm²×10⁻³) measured at P21 (FIG. 4A; 10.8±1.7). The remainder of the transgenic mice (n=9) received an intravitreous injection of 5 ng of rGel in one eye and 5 ng of VEGF/rGel in the fellow eye at P21. At P25, compared to the baseline area of neovascularization per retina at P21, the area of neovascularization per retina in VEGF/rGel mice (FIG. 4C, 2.80±0.98) was significantly less (FIG. 4D). It was also significantly less than the area of neovascularization per retina seen in rGel-injected mice (8.81±1.94, FIG. 4B and FIG. 4D).

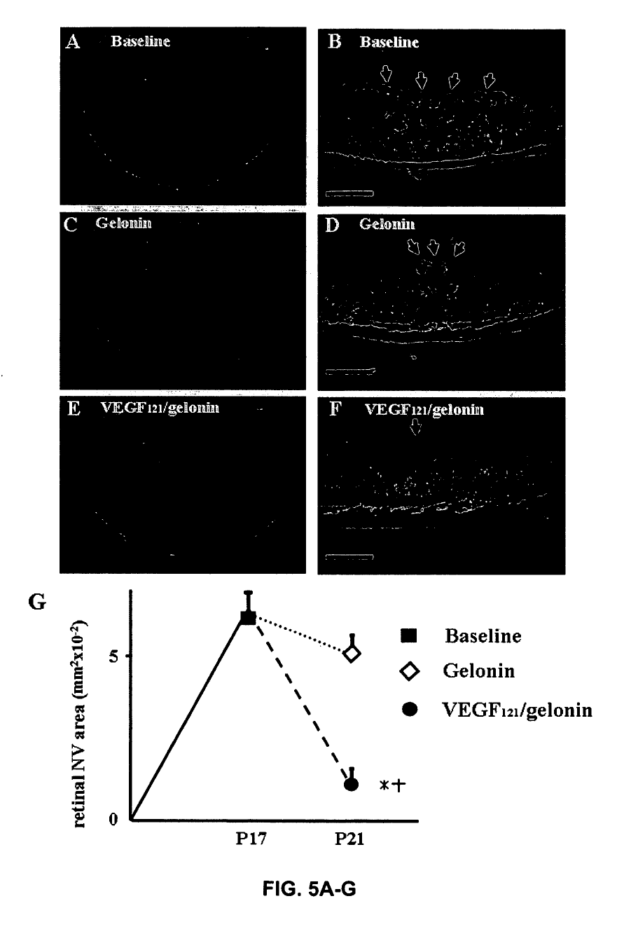

Intravitreous injection of VEGF/rGel causes regression of ischemia induced retinal NV. Mice with oxygen-induced ischemic retinopathy have retinal neovascularization on the surface of the retina similar to that seen in patients with proliferative diabetic retinopathy or retinopathy of prematurity. In this model the amount of neovascularization is fairly stable between P17 and P21 and then regresses spontaneously. There was prominent neovascularization on the surface of the retina at P17 (FIG. 5A and FIG. 5B). Eyes that received an intravitreous injection of rGel at P17 still showed substantial neovascularization on the surface of the retina at P21 (FIG. 5C and FIG. 5D, arrows). However, mice that had been injected with VEGF/rGel at P17 showed almost no identifiable neovascularization at P21 (FIG. 5E and FIG. 5F). Image analysis demonstrated that VEGF/rGel-injected mice had significantly less neovascularization (0.93±0.25 mm²×10⁻²) than mice injected with rGel (5.01±0.46), and significantly less than the baseline amount seen at P17 prior to injection (6.53±0.42, FIG. 5G), indicating that VEGF/rGel induced regression of the retinal neovascularization.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,956,044
U.S. Pat. No. 4,044,126
U.S. Pat. No. 4,045,238
U.S. Pat. No. 4,215,051
U.S. Pat. No. 4,328,803
U.S. Pat. No. 4,364,923
U.S. Pat. No. 4,414,209
U.S. Pat. No. 4,517,295
U.S. Pat. No. 4,554,101
U.S. Pat. No. 4,946,778
U.S. Pat. No. 5,214,080
U.S. Pat. No. 5,229,127
U.S. Pat. No. 5,273,056
U.S. Pat. No. 5,273,056
U.S. Pat. No. 5,273,751
U.S. Pat. No. 5,273,751
U.S. Pat. No. 5,278,126
U.S. Pat. No. 5,282,851
U.S. Pat. No. 5,292,362
U.S. Pat. No. 5,889,155
U.S. Pat. No. 6,692,724
U.S. Appln. 20020114783
U.S. Appln. 20030040496
U.S. Appln. 20030082159
U.S. Appln. 20040248805
U.S. Appln. 20050037967
U.S. application Ser. No. 09/599,152
U.S. application Ser. No. 10/627,763
U.S. application Ser. No. 10/672,142
U.S. application Ser. No. 10/703,405
U.S. application Ser. No. 10/732,919
U.S. application Ser. No. 10/791,692
Adamis et al., Am. J. Ophthalmol., 118:445-450, 1994.
Adamis et al., Arch. Ophthalmol., 114:66-71, 1996.
Aiello et al., N. Engl. J. Med., 331:1480-1487, 1994.
Aiello et al., Proc. Natl. Acad. Sci. USA, 92:10457-10461, 1995.
Anand-Apte et al., Biochem. Cell. Biol., 74:853-862, 1996.
Arora et al., Canc. Res., 59:183-188, 1999.
Bergers et al., Science, 284:808-812, 1999.
Bigg et al., J. Biol. Chem., 272:15496-15500, 1997.
Bird et al., Science, 242:423-426, 1988.
Bittner et al., Methods in Enzymol, 153:516-544, 1987.
Bodanszky et al., J. Antibiot., 29(5):549-53, 1976.
Boehm et al., Nature, 390:404-407, 1997.
Brinkmann and Pastan, Biochim. Biophys. Acta, 1198(1):27-45, 1994.
Brooks et al., Cell, 92:391-400, 1998.
Burbage et al., Leuk Res., 21(7):681-690, 1997.
Canfield et al., Methods Enzymol., 189:418-422, 1990.
Cao et al., J. Clin. Invest., 101(5):1055-1063, 1998.
Cao et al., J. Biol. Chem., 272: 22924-22928, 1997.
Caruthers et al., Nucleic Acids Symp. Ser., (7):215-223, 1980.
Catalysis in Micellar and Macromolecular Systems, Fendler and Fendler (Eds.), NY, Academic Press, 1975.
Chaudhary et al., Proc. Natl. Acad. Sci. USA, 87(3):1066-1070, 1990.
Chow and Kempe, Nucleic Acids Res., 9(12):2807-2817, 1981.
Colbere-Garapin et al., Dev Biol Stand, 50:323-326, 1981.
Colbere-Garapin et al., J. Mol. Biol., 150:1-14, 1981.
Cole et al., J. Immunol. Methods, 78(2):271-278, 1985.
Colloidal Surfactants, Shinoda et al. (Eds.), NY, Academic Press, 1963.
Cote et al., Proc. Natl. Acad. Sci. USA, 80(7):2026-2030, 1983.
Crea and Horn, Nucleic Acids Res., 8(10):2331-2348, 1980.
Creighton, In: Proteins Structures And Molecular Principles, W.H. Freeman and Co., N.Y. 50-60, 1983.
Creighton, In: Proteins Structures And Molecular Principles, W.H. Freeman and Co., NY, 34-49, 1983.
Culver et al., Science, 256(5063): 1550-1552, 1992.
Denekamp, Acta Radiol. Oncol., 23:217-225, 1984.
Denekamp, Eur. J. Clin. Invest., 29:733-736, 1999.
Docherty et al., Nature, 318:666-669, 1985.
El-Gorab et al., Biochim. Biophys. Acta, 306(1):58-66, 1973.
Ferris et al., Arch. Ophthalmol., 102:1640-1642, 1984.
Gaudreault et al., Invest. Ophthalmol. Vis. Sci., 46(2):726-733, 2005.
Gomez et al., Eur. J. Cell. Biol., 74: 111-122, 1997.
Gragoudas et al., N. Eng. J. Med., 351:2805-2816, 2004.
Greene et al., J. Biol. Chem., 271:30375-30380, 1996.
Hartman and Mulligan, Proc. Natl. Acad. Sci. USA, 85:8047-8051, 1988.
Huston et al., Biochemistry, 27(25):8945-8952, 1988.
Ji et al., Biochem. Biophys. Res. Commun., 247:414-419, 1998.
Kirsch et al., Cancer Res., 58:4654-4659, 1998.
Klein et al., Arch. Ophthalmol., 102:520-526, 1984.
Kohler and Milstein, Nature, 256:495-497, 1975.
Kwak et al., Invest. Ophthalmol. Vis. Sci., 41:3158-3164, 2000.
Kyte and Doolittle, J. Mol. Biol., 157(1):105-132, 1982.
Lidor et al., Am. J. Obstet. Gynecol., 177(3):579-585, 1997.
Liu et al., Canc. Res., 62:5470-5475, 2002.
Liu et al., Molec. Canc. Ther., 2:949-959, 2003.
Lowy et al., Cell, 22:817-823, 1980.
Luna et al., Lab. Invest., 75:563-573, 1996.
Luster and Leder, J. Exp. Med., 178:1057-1065, 1993.
Martin et al., Oncogene, 13:569-576, 1996.
Massuda et al., Proc. Natl. Acad. Sci. USA, 94(26):14701-14706, 1997.
Mauceri et al., Nature, 394:287-291, 1998.
McConlogue, Mol. Cell Biol., 6(8):2865-2871, 1986.
Morrison et al., Proc. Natl. Acad. Sci. USA, 81(21):6851-6855, 1984.
Mulligan and Berg, Proc. Natl. Acad. Sci. USA, 78(4):2072-2076, 1981.
Musso et al., J Hepatol., 26:593-605, 1997.
Nambu et al., Invest. Ophthalmol. Vis. Sci., 44:3650-3655, 2003.
Neuberger et al., Nature, 312(5995):604-608, 1984.
Nilsson et al., Canc. Res., 61:711-716, 2001.
O'Hare et al., Proc. Natl. Acad. Sci. USA, 78: 1527, 1981.
O'Reilly et al., Cell, 88:277-85, 1997.
Okamoto et al., Am. J. Pathol., 151:281-91, 1997.
Ozaki et al., Am. J. Pathol., 156:679-707, 2000.
Pasqualini et al., Nat. Biotechnol., 15:542-546, 1997.
Ramakrishnan et al., Canc. Res., 56:1324-1330, 1996.
Ran et al., Canc. Res., 58:4646-4653, 1998.
Remington's Pharmaceutical Sciences, 15^{th} ed., pages 1035-1038 and 1570-1580, Mack Publishing Company, Easton, Pa., 1980.
Ruther et al., EMBO J, 2(10):1791-4, 1983.
Saishin et al., J. Cell. Physiol., 195:241-248, 2003.
Sambrook et al., In: Molecular cloning, Cold Spring Harbor Laboratory Press, Cold Spring Harbor, N.Y., 2001.
Santerre et al., Gene, 30: 147-156, 1984.
Sasaki et al., J. Mol. Biol., 301(5):1179-1190, 2000.
Seo et al., Am. J. Pathol., 154:1743-1753, 1999.
Sgadari et al., Blood, 89:2635-2643, 1997.
Sgadari et al., Proc. Natl. Acad. Sci. USA, 93:13791-13796, 1996.
Smith and Moss, Gene, 25(1):21-8, 1983.
Smith et al., Invest. Ophthalmol. Vis. Sci., 35:101-111, 1994.
Stetler-Stevenson et al., J. Biol. Chem., 265:13933-13938, 1990.
Szybalski and Szybalski, Proc. Natl. Acad. Sci. USA, 48:2026-2034, 1962.
Takeda et al., Nature, 314(6010):452-454, 1985.
Tanaka et al., Nat. Med., 4(1):31-36, 1998.
Tannenbaum et al., J. Immunol., 161:927-932, 1998.
Thorpe, Clin. Canc. Res., 10:415-427, 2004.
Tobe et al., Invest. Ophthalmol. Vis. Sci., 39:180-8, 1998.
Valente et al., Int. J. Cancer, 75:246-253, 1998.
Van Heeke and Schuster, J. Biol. Chem., 264(33):19475-19477, 1989.
Veenendaal et al., Proc. Natil. Acad. Sci. USA, 99:7866-7871, 2002.
Verbeke and Molenberghs, In: Linear Mixed Models for Longitudinal Data, 93-120, Springer-Verlag, Inc., NY, 2000
Wang et al., Oncogene, 14:2767-2774, 1997.
Wigler et al., Cell, 11(1):223-232, 1977.
Wigler et al., Proc. Natl. Acad. Sci. USA, 77(6):3567-3570, 1980.
Witmer et al., Prog. Retin. Eye Res., 22(1):1-29, 2003.
Zetter, Annu. Rev. Med., 49:407-424, 1998.

What is claimed is:

1. A method of treating or preventing eye disease in a subject, comprising administering to the subject a therapeutically effective amount of a pharmaceutical composition comprising a polypeptide having a vascular endothelial targeting amino acid sequence and a cytotoxic amino acid sequence which results in treatment or prevention of eye disease in the subject.

2. The method of claim 1, wherein the polypeptide further comprises a linker between the vascular endothelial targeting amino acid sequence and the cytotoxic amino acid sequence.

3. The method of claim 2, wherein the linker is G₄S, (G₄S)₂, (G₄S)₃, 218 linker, an enzymatically cleavable linker, or a pH cleavable linker.

4. The method of claim 1, wherein the vascular endothelial targeting amino acid sequence is selected from the group consisting of VEGF, FGF, integrin, fibronectin, I-CAM, PDGF, or an antibody to a molecule expressed on the surface of a vascular endothelial cell.

5. The method of claim 4, wherein VEGF is an isoform selected from the group consisting of VEGF₁₂₁, VEGF₁₆₅, VEGF₁₈₉, and VEGF₂₀₆.

6. The method of claim 5, wherein the VEGF is VEGF₁₂₁.

7. The method of claim 6, wherein the VEGF sequence is selected from the group consisting of SEQ ID NOs:4-10.

8. The method of claim 1, wherein the cytotoxic amino acid sequence is a toxin.

9. The method of claim 8, wherein the toxin is a ribosome inactivating protein (RIP).

10. The method of claim 9, wherein the ribosome inactivating protein is selected from the group consisting of gelonin, maize RIP, saporin, ricin, ricin A chain, barley RIP, momordin, alpha-momorcharin, beta-momorcharin, Shiga-like RIP, and a-sarcin.

11. The method of claim 8, wherein the toxin is selected from the group consisting of abrin, an aquatic-derived cytotoxin, Pseudomonas exotoxin, a DNA synthesis inhibitor, a RNA synthesis inhibitor, a prodrug, a light-activated porphyrin, trichosanthin, tritin, pokeweed antiviral protein, mirabilis antiviral protein (MAP), Dianthin 32, Dianthin 30, bryodin, shiga, diphtheria toxin, diphtheria toxin A chain, dodecandrin, tricokirin, bryodin, and luffin.

12. The method of claim 1, wherein the cytotoxic amino acid sequence is an anti-angiogenic amino acid sequence selected from the group consisting of TIMP-1, TIMP-2, TIMP-3, TIMP-4, endostatin, angiostatin, endostatin XVIII, endostatin XV, the C-terminal hemopexin domain of matrix metalloproteinase-2, the kringle 5 domain of human plasminogen, the monokine-induced by interferon-gamma (Mig), the interferon-alpha inducible protein 10 (IP10), soluble FLT-1 (fms-like tyrosine kinase 1 receptor), and kinase insert domain 15 receptor (KDR).

13. The method of claim 1, wherein the cytotoxic amino acid sequence induces apoptosis.

14. The method of claim 13, wherein the cytotoxic amino acid sequence is selected from the group consisting of granzyme B, Bax, TNF-α, TNF-β, TGF-β, IL-12, IL-3, IL-24, IL-18, TRAIL, IFN-α, INF-β, IFN-γ, a Bcl protein, Fas ligand, or a caspase.

15. The method of claim 1, wherein the subject is a mammal.

16. The method of claim 15, wherein the mammal is a human.

17. The method of claim 1, wherein the eye disease is associated with neovascularization.

18. The method of claim 17, wherein the neovascularization is retinal neovascularization, choroidal neovascularization, or other ophthalmic neovascularization.

19. The method of claim 1, wherein the eye disease is age-related macular degeneration, diabetic retinopathy, proliferative vitreoretinopathy, or an ophthalmic tumor.

20. The method of claim 19, wherein the ophthalmic tumor is a choroidal melanoma, a retinoblastoma, a metastatic tumor, or a uveal melanoma.

21. The method of claim 1, wherein the composition is administered intravascularly.

22. The method of claim 1, wherein the composition is administered intraocularly.

23. The method of claim 22, wherein intraocular administration is by intravitreal administration, administration into the anterior chamber, or administration into an intraocular tumor.

24. The method of claim 1, wherein the subject has neovascularization secondary to age-related macular degeneration and is administered a fusion protein of VEGF₁₂₁ and recombinant gelonin by intravitreal administration.

25. The method of claim 24, wherein between about 0.5 ng and about 10 ng of the fusion protein is administered intravitreally.

26. The method of claim 25, wherein between about 1 ng and about 4 ng of the fusion protein is administered intravitreally.

27. The method of claim 1, further comprising administering the pharmaceutical composition more than once.

28. The method of claim 1, further comprising treatment with other eye therapy.

29. The method of claim 28, wherein the other therapy is oral therapy, topical therapy, intraocular therapy, laser photocoagulation, cryotherapy, radiation therapy, surgical therapy, gene therapy, and immunotherapy.

30. The method of claim 1, further comprising identifying a patient in need of such therapy.

31. A method of treating or preventing an eye disease associated with neovascularization in a subject comprising intraocularly administering to the subject a therapeutically effective amount of a composition comprising a polypeptide having a vascular endothelial targeting amino acid sequence and a cytotoxic amino acid sequence, wherein the eye disease is prevented or treated.

32. The method of claim 31, wherein the polypeptide further comprises a linker between the vascular endothelia targeting amino acid sequence and the cytotoxic amino acid sequence.

33. The method of claim 32, wherein the linker is G₄S, (G₄S)₂, (G₄S)₃, 218 linker, an enzymatically cleavable linker, or a pH cleavable linker.

34. The method of claim 31, wherein the vascular endothelial targeting amino acid sequence is selected from the group consisting of VEGF, FGF, integrin, fibronectin, I-CAM, PDGF, or an antibody to a molecule expressed on the surface of a vascular endothelial cell.

35. The method of claim 34, wherein VEGF is an isoform selected from the group consisting of VEGF₁₂₁, VEGF₁₆₅, VEGF₁₈₉, and VEGF₂₀₆.

36. The method of claim 35, wherein the VEGF is VEGF₁₂₁.

37. The method of claim 36, wherein the VEGF sequence is selected from the group consisting of SEQ ID NOs:4-10.

38. The method of claim 31, wherein the cytotoxic amino acid sequence is a toxin.

39. The method of claim 38, wherein the toxin is a ribosome inactivating protein (RIP).

40. The method of claim 39, wherein the ribosome inactivating protein is selected from the group consisting of gelonin, maize RIP, saporin, ricin, ricin A chain, barley RIP, momordin, alpha-momorcharin, beta-momorcharin, Shiga-like RIP, and a-sarcin.

41. The method of claim 38, wherein the toxin is selected from the group consisting of abrin, an aquatic-derived cytotoxin, Pseudomonas exotoxin, a DNA synthesis inhibitor, a RNA synthesis inhibitor, a prodrug, a light-activated porphyrin, trichosanthin, tritin, pokeweed antiviral protein, mirabilis antiviral protein (MAP), Dianthin 32, Dianthin 30, bryodin, shiga, diphtheria toxin, diphtheria toxin A chain, dodecandrin, tricokirin, bryodin, and luffin.

42. The method of claim 31, wherein the cytotoxic amino acid sequence induces apoptosis.

43. The method of claim 42, wherein the cytotoxic amino acid sequence is selected from the group consisting of granzyme B, Bax, TNF-α, TNF-β, TGF-β, IL-12, IL-3, IL-24, IL-18, TRAIL, IFN-α, INF-β, IFN-γ, a Bcl protein, Fas ligand, or a caspase.

44. The method of claim 31, wherein the cytotoxic amino acid sequence is an anti-angiogenic amino acid sequence selected from the group consisting of TIMP-1, TIMP-2, TIMP-3, TIMP-4, endostatin, angiostatin, endostatin XVIII, endostatin XV, the C-terminal hemopexin domain of matrix metalloproteinase-2, the kringle 5 domain of human plasminogen, the monokine-induced by interferon-gamma (Mig), the interferon-alpha inducible protein 10 (IP10), soluble FLT-1 (fms-like tyrosine kinase 1 receptor), and kinase insert domain 15 receptor (KDR).

45. The method of claim 31, wherein the subject is a mammal.

46. The method of claim 45, wherein the mammal is a human.

47. The method of claim 31, wherein the neovascularization is retinal neovascularization, choroidal neovascularization, or other ophthalmic neovascularization.

48. The method of claim 31, wherein the eye disease is age-related macular degeneration, diabetic retinopathy, proliferative vitreoretinopathy, or an ophthalmic tumor.

49. The method of claim 48, wherein the ophthalmic tumor is a choroidal melanoma, a retinoblastoma, a metastatic tumor, or a uveal melanoma.

50. The method of claim 31, wherein intraocular administration is by intravitreal administration, administration into the anterior chamber, or administration into an intraocular tumor.

51. The method of claim 31, wherein the subject has neovascularization secondary to age-related macular degeneration and is administered a fusion protein of VEGF₁₂₁ and recombinant gelonin by intravitreal administration.

52. The method of claim 51, wherein between about 0.5 ng and about 10 ng of the fusion protein is administered intravitreally.

53. The method of claim 52, wherein between about 1 ng and about 4 ng of the fusion protein is administered intravitreally.

54. The method of claim 31, further comprising administering the pharmaceutical composition more than once.

55. The method of claim 31, further comprising treatment with other eye therapy.

56. The method of claim 55, wherein the other therapy is oral therapy, topical therapy, intraocular therapy, laser photocoagulation, cryotherapy, radiation therapy, surgical therapy, gene therapy, and immunotherapy.

57. The method of claim 31, further comprising identifying a patient in need of such therapy.


SEQUENCE LISTING (10 sequences)

SEQ ID NO: 1 - protein, 5 aa, Artificial Sequence
  Description of Artificial Sequence  Synthetic Peptide
GGGGS

SEQ ID NO: 2 - protein, 14 aa, Artificial Sequence
  Description of Artificial Sequence  Synthetic Peptide
EGKSSGSGSESKVD

SEQ ID NO: 3 - protein, 18 aa, Artificial Sequence
  Description of Artificial Sequence  Synthetic Peptide
KESGSVSSEQLAQFRSLD

SEQ ID NO: 4 - protein, 121 aa, Artificial Sequence
  Description of Artificial Sequence  Synthetic Peptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENCDKPRR

SEQ ID NO: 5 - protein, 206 aa, Artificial Sequence
  Description of Artificial Sequence  Synthetic Peptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 6 - protein, 189 aa, Artificial Sequence
  Description of Artificial Sequence  Synthetic Peptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 7 - protein, 183 aa, Artificial Sequence
  Description of Artificial Sequence  Synthetic Peptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 8 - protein, 165 aa, Artificial Sequence
  Description of Artificial Sequence  Synthetic Peptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 9 - protein, 148 aa, Artificial Sequence
  Description of Artificial Sequence  Synthetic Peptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKM

SEQ ID NO: 10 - protein, 145 aa, Artificial Sequence
  Description of Artificial Sequence  Synthetic Peptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVCDKPRR